SEQUENCE LISTING (41 sequences)

SEQ ID NO: 1 - protein, 8 aa, Artificial Sequence
  Synthetic
GFSFNSFW

SEQ ID NO: 2 - protein, 8 aa, Artificial Sequence
  Synthetic
TNNEGTTT

SEQ ID NO: 3 - protein, 9 aa, Artificial Sequence
  Synthetic
ARGDGGLDD

SEQ ID NO: 4 - protein, 116 aa, Artificial Sequence
  Synthetic
EVQLVESGGGLVQPGGSLRLSCSASGFSFNSFWMHWVRQVPGKGLVWISFTNNEGTTTAYADSVRGRFIISRDNAKNTLYLEMNNLRGEDTAVYYCARGDGGLDDWGQGTLVTVSS

SEQ ID NO: 5 - protein, 12 aa, Artificial Sequence
  Synthetic
QSIFRTSRNKNL

SEQ ID NO: 6 - protein, 9 aa, Artificial Sequence
  Synthetic
QQYFSPPYT

SEQ ID NO: 7 - protein, 113 aa, Artificial Sequence
  Synthetic
DIQLTQSPDSLAVSLGERATINCKSSQSIFRTSRNKNLLNWYQQRPGQPPRLLIHWASTRKSGVPDRFSGSGFGTDFTLTITSLQAEDVAIYYCQQYFSPPYTFGQGTKLEIK

SEQ ID NO: 8 - protein, 8 aa, Artificial Sequence
  Synthetic
GFSLTNYG

SEQ ID NO: 9 - protein, 7 aa, Artificial Sequence
  Synthetic
IWSGGNT

SEQ ID NO: 10 - protein, 12 aa, Artificial Sequence
  Synthetic
ARDRYDVRAFDY

SEQ ID NO: 11 - protein, 118 aa, Artificial Sequence
  Synthetic
QVQLKQSGPGLVQPSQSLSITCTVSGFSLTNYGVHWVRQSPGKGLEWLGVIWSGGNTDYNAAFISRLSISKDNSKSQVFFKMNSLQADDTAIYYCARDRYDVRAFDYWGQGTTLTVSS

SEQ ID NO: 12 - protein, 6 aa, Artificial Sequence
  Synthetic
QNVRTA

SEQ ID NO: 13 - protein, 9 aa, Artificial Sequence
  Synthetic
LQHWNYLYT

SEQ ID NO: 14 - protein, 108 aa, Artificial Sequence
  Synthetic
DIVMTQSQKFMSTSVGDRVSITCKASQNVRTAVAWYQQKPGQSPKALIYLASNRHTGVPDRFTGSGSGTDFTLTISNVQSEDLADYFCLQHWNYLYTFGGGTKLEIKK

SEQ ID NO: 15 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 16 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHSALHNHYTQKSLSLSPGK

SEQ ID NO: 18 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHFALHNHYTQKSLSLSPGK

SEQ ID NO: 19 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHTALHNHYTQKSLSLSPGK

SEQ ID NO: 20 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRQPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRYPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKWLSLSPGK

SEQ ID NO: 24 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKYLSLSPGK

SEQ ID NO: 25 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHGALHNHYTQKSLSLSPGK

SEQ ID NO: 26 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRKPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHSALHNHYTQKSLSLSPGK

SEQ ID NO: 28 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHFALHNHYTQKSLSLSPGK

SEQ ID NO: 29 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHTALHNHYTQKSLSLSPGK

SEQ ID NO: 30 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRQPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRRPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRYPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKWLSLSPGK

SEQ ID NO: 34 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKYLSLSPGK

SEQ ID NO: 35 - protein, 8 aa, Artificial Sequence
  Synthetic
GYTFTSYD

SEQ ID NO: 36 - protein, 8 aa, Artificial Sequence
  Synthetic
MNPNSGNT

SEQ ID NO: 37 - protein, 19 aa, Artificial Sequence
  Synthetic
ATERWSKDTGHYYYYGMDV

SEQ ID NO: 38 - protein, 455 aa, Artificial Sequence
  Synthetic
QVQLQQSGAEVKKPGASVKVSCRASGYTFTSYDINWVRQAPGQGLEWMGWMNPNSGNTNYAQRFQGRLTMTKNTSINTAYMELSSLRSEDTAVYYCATERWSKDTGHYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 7 aa, Artificial Sequence
  Synthetic
QSVSSSY

SEQ ID NO: 40 - protein, 9 aa, Artificial Sequence
  Synthetic
QKYGSTPRP

SEQ ID NO: 41 - protein, 215 aa, Artificial Sequence
  Synthetic
ETTLTQSPGTLSLSPGERATLSCRASQSVSSSYLAWYQQKPGQAPKVLIYDASSRATGIPDRFSGSGSGTDFTLTISRLEPEDFAVYYCQKYGSTPRPFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0332801 A1
(43) Pub. Date: Oct. 20, 2022

(54) ANTIBODIES AND METHODS OF USE THEREOF IN TREATMENT OF INFECTIOUS DISEASE

(71) Applicant: GENMAB B.V., Utrecht (NL)

(72) Inventors: Annemarie KUIPERS, Utrecht (NL); Kok VAN KESSEL, Utrecht (NL); Frank BEURSKENS, Utrecht (NL); Rob DE JONG, Utrecht (NL); Kristin STRUMANE, Werkhoven (NL); Janine SCHUURMAN, Diemen (NL); Paul PARREN, Utrecht (NL); Jos VAN STRIJP, Utrecht (NL); Suzan ROOIJAKKERS, Utrecht (NL)

(21) Appl. No.: 17/534,712

(22) Filed: Nov. 24, 2021

Related U.S. Application Data
(63) Continuation of application No. 16/301,584, filed on Nov. 14, 2018, now abandoned.

(30) Foreign Application Priority Data
May 18, 2016 (DK) PA 2016 00305

(51) Int. Cl. C07K 16/12; A61P 31/04

(52) U.S. Cl. CPC C07K 16/1271; A61P 31/04; A61K 2039/507

(57) ABSTRACT

The present invention relates to antibody molecules that bind to Wall Teichoic Acid (WTA) or Capsular Polysaccharides (CP) such as Capsular Polysaccharides type 5 (CP5). The invention relates in particular to antibody molecules of the IgG isotype having a mutation in the Fc domain that enhances clustering of IgG molecules after target binding. The invention also relates to pharmaceutical compositions containing these molecules and the treatment of infectious diseases using these compositions

### CROSS-REFERENCE TO RELATED APPLICATIONS

This application is a continuation of U.S. patent application Ser. No. 16/301,584, filed on Nov. 14, 2018, which is a 35 U.S.C. 371 national stage filing of International Application No. PCT/EP2017/061879, filed on May 17, 2017, which claims priority to Danish Patent Application No. PA 2016 00305, filed on May 18, 2016. The contents of the aforementioned applications are hereby incorporated by reference.

### SEQUENCE LISTING

The instant application contains a Sequence Listing which has been submitted electronically in ASCII format and is hereby incorporated by reference in its entirety. Said ASCII copy, created on Nov. 24, 2021, is named GMI_177USCN_Sequence_Listing.txt and is 71,355 bytes in size.

### FIELD OF THE INVENTION

The present invention relates to an antibody that bind to Wall Teichoic Acid (WTA) or Capsular Polysaccharides (CP), such as Capsular Polysaccharides type 5 (CP5). The invention relates in particular to antibody molecules of the IgG isotype having a mutation in the Fc region that enhances clustering of IgG molecules after target binding. The invention also relates to pharmaceutical compositions containing these molecules and the treatment of infectious diseases using these compositions.

### BACKGROUND OF THE INVENTION

Pathogenic bacteria are a substantial cause of sickness and death in both humans and animals.

Staphylococcus aureus (S. aureus) is a leading human pathogen that causes very serious infections. S. aureus can harmlessly colonize around 30% of healthy humans but also cause life-threatening diseases in both hospital and community settings. In hospitals, S. aureus is one of the most significant causes of infections ranging from superficial wound infections to severe conditions like sepsis, endocarditis and necrotizing pneumonia. The incidence of both hospital and community hypervirulent S. aureus strains resistant to beta-lactam antibiotics (MRSA) and multi-resistant S. aureus is growing. In humans, host clearance of S. aureus critically depends on proper engulfment and intracellular killing by phagocytic cells such as neutrophils and macrophages. In order to effectively engulf S. aureus, phagocytic cells depend on the complement system, a large protein network in plasma. Upon contact with bacteria, the complement proteins organize into a cascade of proteolytic events that eventually results in massive labeling of bacterial surfaces with complement proteins C3b and iC3b. These ‘opsonic’ C3b/iC3b molecules potently enhance phagocytosis efficiency via interaction with complement receptors (CD35, CD11b/CD18) on phagocytic cells. The classical complement pathway is an important route to trigger the complement cascade on bacteria. This pathway is initiated by C1q, a hexamer of globular heads that bind bacterium-bound antibodies. Upon binding, C1q activates its associated enzyme C1s to cleave components C4 and C2 to form a C3 convertase enzyme (C4b2a). This C3 convertase, attached to the surface via C4b, rapidly catalyzes the covalent deposition of C3b molecules onto the bacterial surface.

Different antibody-based biological agents have been evaluated for their clinical efficacy (reviewed in Sause et. al., 2015 Trens in Pharmacological Sciences), including pooled human immunoglobulin (altastaph, veronate), an antibody fragment against anti-GrfA (aurograb), and monoclonal antibodies (anti-CIfA, tefibazumab; anti-lipoteichoic acid, pagibaximab; anti-PNAG, F598). Other antibody-based biologics against MRSA that has been described include monoclonal antibodies against different target molecules (including leukotoxins, alpha hemolysin, glucosaminidase subunit of AtI, IsaA, Protein A), and an anti-wall teichoic acid (WTA) mAb-drug conjugate (ADC) (reviewed in Sause et. al., 2015 Trens in Pharmacological Sciences). Also an IgM against a capsule antigen has been described (WO2009140236).

WO2014/193722 and WO2014/194247 discloses anti-wall teichoic acid (anti-WTA) antibodies conjugated to antibiotics and uses of the antibody-antibiotic conjugate in treatment of infectious diseases.

WO2013/004842 discloses polypeptides with a variant Fc domain and antibodies or polypeptides having modified effector functions resulting from modifications in the Fc domain.

WO2014/108198 discloses Fc containing polypeptides with increased CDC resulting from modifications in the Fc-domain

However, there is a need for improving antibody therapies against infectious diseases such as bacterial infections and it is desirable for the antibody-based formats to preserve a pharmacokinetic (PK) profile close to that of regular IgG and a predictable safety profile, which is often not the case with antibody fragment-based construct or antibodies conjugated to various other toxins.

The present invention provides for antibodies for use in the treatment of infectious diseases, such as antibodies with binding specificities to Wall Teichoic Acid (WTA), Capsular polysacharrides (CP), such as Capsular Polysacharrides type 5 (CP5), with modified Fc regions. Antibodies of the invention with modified Fc regions show enhanced phagocytic activity compared to a parent antibody with the same antigen specificity but without a modification in the Fc region.

### SUMMARY OF THE INVENTION

The inventors of the present invention have found that introduction of a specific point mutation in the Fc region of antibodies binding to WTA or capsular polysaccharide molecules e.g. CP5, which are components of the cell wall of bacteria, significantly enhances the potency of the antibody to induce FcγR-independent clustering of the antibody after binding to the target on the bacterial cell surface. The inventors have also found that the antibodies of the invention enhance complement activation and phagocytosis and bacterial cell clearance.

The object of the present invention is to provide a modified anti-WTA antibody or a modified anti-CP antibody, such as a modified anti-CP5 antibody, suitable for use in treatment of infectious diseases. It is a further object of the invention to provide modified antibodies as presented herein for the use in treatment of bacterial infections. Such a modified anti-WTA antibody or an anti-CP antibody, such as an anti-CP5 antibody comprises a mutation in the Fc region. A further object of the present invention is to provide a composition suitable for the treatment of bacterial infections comprising one or more modified anti-WTA antibodies or one or more anti-CP antibodies, such as one or more anti-CP5 antibodies. Such composition as described herein comprises at least one anti-WTA antibody or at least one anti-CP antibody e.g. at least one anti-CP5 antibody according to the invention, and more preferably the composition comprises two or more anti-WTA antibodies or anti-CP antibodies, such as anti-CP5 antibodies according to the invention.

The present invention provides an antibody comprising an Fc region of a human immunoglobulin IgG and an antigen binding region binding to WTA or CP, such as anti-WTA antibodies or anti-CP antibodies, such as anti-CP5 antibodies, wherein the Fc region comprises a mutation corresponding to position E430, E345 or S440 in human IgG1 according to EU numbering. That is an antibody according to the present invention comprises an Fc region of a human immunoglobulin G, with a mutation of an amino acid at a position corresponding to E430, E345 or S440 in human IgG1 according to EU numbering. That is the amino acid at a position corresponding to E430, E345 or S440 in human IgG1 corresponds to the amino acid at position E430, E345 or S440 in amino acid sequence of human IgG1 according to EU numbering.

That is, the inventors of the present invention have in a first aspect of the invention found that an anti-WTA antibody or an anti-CP5 antibody of the invention increases phagocytosis of bacterial cells expressing WTA or CP5, when compared to an anti-WTA or an anti-CP5, without a mutation corresponding to position E430, E345 or S440 of human IgG1, EU numbering. That is, the anti-WTA antibody or anti-CP antibody, such as an anti-CP5 antibody, of the present invention is suitable for the treatment of infectious diseases. Infectious diseases such as bacteria expressing WTA or CP such as CP5 are suitable for treatment with an antibody of the present invention. Further, diseases caused by gram positive bacteria such as skin and soft tissue infections (SSTI's), pneumonia, purulent cellulitis meningitis, cystic fibrosis, osteomyelitis, endocarditis, toxic shock syndrome device-related infections, bacteremia and sepsis can be treated by antibodies of the invention. Further, diseases caused by Staphylococcus aureus such as skin and soft tissue infections (SSTI's), pneumonia, bacteremia, endocarditis and osteomyelitis can be treated by antibodies of the invention. Further, diseases caused by Staphylococcus warneri such as vertebral discitis, urinary tract infection, meningitis, orthopedic infections, ventricular shunt infections and endocarditis can be treated by antibodies according to the invention.

In one embodiment the anti-WTA antibody comprises an Fc region of human IgG, wherein the Fc region comprises a mutation in an amino acid position corresponding to E430 in human IgG1, according to EU numbering.

In one embodiment the anti-WTA antibody comprises an Fc region of human IgG, wherein the Fc region comprises a mutation in an amino acid position corresponding to E345 in human IgG1.

In one embodiment the anti-WTA antibody comprises an Fc region of human IgG, wherein the Fc region comprises a mutation in an amino acid position corresponding to S440 in human IgG1.

In one embodiment of the present invention the anti-WTA, antibody comprises an Fc region of a human immunoglobulin IgG and an antigen binding region binding to WTA, wherein the Fc region comprises a mutation corresponding to E430G or E345K in human IgG1 according to EU numbering. In one embodiment the anti-WTA antibody is an anti-WTA-α antibody. In another embodiment the anti-WTA antibody is an anti-WTA-β antibody. That is an antibody according to the present invention comprises an Fc region of a human immunoglobulin G, with a mutation corresponding to amino acid position E430, E345 or S440 in human IgG1 according to EU numbering.

In one embodiment of the present invention the anti-CP antibody comprises an Fc region of a human immunoglobulin IgG and an antigen binding region binding to CP, wherein the Fc region comprises a mutation corresponding to E430G or E345K in human IgG1 according to EU numbering.

In one embodiment the anti-CP5 antibody comprises an Fc region of human IgG, wherein the Fc region comprises a mutation in an amino acid position corresponding to E430 in human IgG1.

In one embodiment the anti-CP5 antibody comprises an Fc region of human IgG, wherein the Fc region comprises a mutation in an amino acid position corresponding to E345 in human IgG1.

In one embodiment the anti-CP5 antibody comprises an Fc region of human IgG, wherein the Fc region comprises a mutation in an amino acid position corresponding to S440 in human IgG1.

In one embodiment of the present invention the anti-CP5 antibody comprises an Fc region of a human immunoglobulin IgG and an antigen binding region binding to CP5, wherein the Fc region comprises a mutation corresponding to E430G or E345K in human IgG1 according to EU numbering.

In one aspect the present invention provides for an anti-WTA antibody, an anti-CP antibody, an anti-CP5 antibody, wherein the Fc region comprises a mutation in an amino acid position corresponding to E430, E345 or S440 in human IgG1, for use as a medicament.

In one aspect the present invention provides for an anti-WTA antibody, an anti-CP antibody, an anti-CP5 antibody, wherein the Fc region comprises a mutation in an amino acid position corresponding to E430, E345 or S440 in human IgG1, for use in treatment of infectious disease.

In one aspect the invention provides a composition comprising one or more antibodies of the invention. The composition may comprise on or more of the following group of antibodies consisting of: an anti-WTA-α antibody, anti-WTA-β antibody, an anti-CP antibody and an anti-CP5 antibody.

In another aspect the invention provides for an antibody or a composition as described herein for use as a medicament.

In one aspect the invention provides for an antibody or a composition as described herein for use in treatment of an infection caused by gram positive bacteria.

In yet another aspect the invention provides a method of treating an individual having an infectious disease comprising administering to said individual an effective amount of said antibody or composition as described herein.

In another aspect the invention provides the use of an antibody or a composition as described herein for the manufacture of a medicament for treatment of a disease. In one embodiment the invention provides the use of an antibody or a composition as described herein for the manufacture of a medicament for treatment of an infectious disease.

### BRIEF DESCRIPTION OF THE DRAWINGS

FIGS. 1A-1D show deposition of complement activation products on S. aureus bacteria by naturally occurring antibodies in the presence or absence of Fc-binding peptide DCAWHLGELVWCT (Fc-III) or control peptides. Wood 46 bacteria were opsonized in either a concentration series of pooled normal human serum (NHS) pre-incubated with 20 μg/mL peptide (FIGS. 1A and 1B) or 1% serum pre-incubated with a peptide concentration series (FIGS. 1C and 1D). C4b deposition (FIGS. 1A and 1C) and C3b deposition (FIGS. 1B and 1D) were measured by FACS analysis. Mean fluorescence intensity (MFI) is shown. Graphs present the mean+/−the standard error of the mean (SEM) of two (FIGS. 1C and 1D) or three (FIGS. 1A and 1B) separate experiments.

FIG. 2 shows neutrophil-mediated phagocytic uptake of FITC-labeled S. aureus Wood 46 bacteria after opsonization with pooled NHS in the presence or absence of the indicated concentrations of Fc-binding Fc-III peptide or a non-binding control peptide. Phagocytosis is represented by the MFI of gated neutrophils as measured by FACS analysis. HI serum, heat-inactivated serum; Buffer, no peptide control. Graphs present the mean+/−SEM of three separate experiments.

FIG. 3 shows the induction of C5a release after incubation of Wood 46 bacteria with antibodies against S. aureus present in pooled NHS in the presence or absence of Fc-III peptide or non-binding control peptides. C5a release was measured in a C5a reporter assay. Fluorescence was determined by FACS analysis and presented as MFI relative to buffer control sample without peptide, which was set to 1.0. Bars represent the Mean±standard deviation (sd) of two separate experiments.

FIG. 4 shows binding of 1 μg/mL anti-WTA IgG1-S4497 and anti-Clfa IgG1-T1-2-F405L to S. aureus strains Wood 46, USA300, 8325-4 and COL as measured by FACS analysis. Antibody binding is represented as MFI+/−SEM of two separate experiments.

FIGS. 5A-5E show the effect of introducing the hexamerization enhancing mutation E430G in anti-WTA IgG1-S4497 on binding, complement deposition and neutrophil-mediated phagocytic uptake of S. aureus. (FIG. 5A) binding of IgG1-S4497-E430G to Wood 46 and USA300 as determined by FACS analysis. Binding is presented as MFI relative to binding of wild type IgG1-S4497. (FIGS. 5B and 5C) C4b (FIG. 5B) and C3b (FIG. 5C) deposition on Wood 46 after binding of IgG1-S4497 or IgG1-S4497-E430G in a purified classical pathway system as determined by FACS analysis. (FIGS. 5D and 5E) Neutrophil-mediated phagocytic uptake of GFP-labeled S. aureus Wood 46 bacteria after binding of IgG1-S4497 or IgG1-S4497-E430G in 3% IgG/IgM-depleted serum (FIG. 5D) or in 0.1% NHS (FIG. 5E). Phagocytic uptake is represented by the MFI of gated neutrophils as measured by FACS analysis and expressed relative to the value for WT IgG1 (no serum) at 1.25 μg/mL. The IgG1-b12 mAb against HIV gp120 was used as a non-binding isotype control mAb. Graphs present the Mean+/−SEM of two (FIG. 5C) or three separate experiments.

FIGS. 6A-6C show the effect of the K439E and S440K mutations in anti-WTA IgG1-S4497 on binding and complement deposition on S. aureus. (FIG. 6A) binding of IgG1-S4497-K439E to Wood 46 and USA300 as determined by FACS analysis. Binding is presented as MFI relative to binding of WT IgG1-S4497. (FIGS. 6B and 6C) C4b (FIG. 6B) and C3b (FIG. 6C) deposition on Wood 46 after binding of the single self-repulsing antibodies IgG1-S4497-K439E and IgG1-S4497-S440K or the combination IgG1-S4497-K439E+IgG1-S4497-S440K.

C4b and C3b deposition were analyzed in a purified classical pathway system and determined by FACS analysis. Graphs represent the Mean+/−SEM of two (FIG. 6C) or three separate experiments.

FIG. 7 shows neutrophil-mediated phagocytic uptake of GFP-labeled S. aureus Wood 46 bacteria after binding of the indicated anti-WTA IgG1 and IgG2 antibodies in pooled NHS. Phagocytic uptake is represented by the MFI of gated neutrophils as measured by FACS analysis and expressed relative to the value for WT IgG1 (no serum) at 10 μg/mL. Graphs represent Mean+/−SEM derived from four separate experiments.

FIGS. 8A-8F show the effect of introducing the hexamerization enhancing mutation E430G in IgG1-CP5 on binding, complement deposition and neutrophil-mediated phagocytic uptake of S. aureus. (FIG. 8A) binding of IgG1-CP5 to Reynolds CP5, Reynolds CP-, COL and Newman−/− as determined by FACS analysis. Binding is presented as MFI. (FIG. 8B) Binding of IgG1-CP5-E430G to Reynolds CP5 and Newman−/− as determined by FACS analysis. Binding is presented as MFI relative to binding of WT IgG1-CP5. (FIGS. 8C and 8D) C4b (FIG. 8C) and C3b (FIG. 8D) deposition on Reynolds CP5 after binding of IgG1-CP5 or IgG1-CP5-E430G in NHS or heat-inactivated (HI) serum as determined by FACS analysis. (FIGS. 8E and 8F) Neutrophil-mediated phagocytic uptake of GFP-labeled S. aureus Reynolds CP5 bacteria after binding with IgG1-CP5 or IgG1-CP5-E430G in the presence (FIG. 8F) or absence (FIG. 8E) of competing Fc-III peptide or Fc-III scrambled 1 peptide in the presence of 3% pooled NHS. Phagocytic uptake is represented by the MFI of gated neutrophils as measured by FACS analysis and expressed relative to the value for WT at 10 μg/mL. Error bars in the graphs present the Mean+/−SEM of multiple separate experiments (two in FIGS. 8A, 8B, and 8D; three or four in FIG. 8F and four in FIG. 8E).

FIG. 9 shows the effect of introducing the hexamerization enhancing mutation E430G in anti-WTA IgG1-S4497 on phagocytic killing of S. aureus bacteria. Phagocytic kill of Wood 46 bacteria is represented by the percentage living bacteria after binding of IgG1-S4497 or IgG1-S4497-E430G in 1% IgG/IgM-depleted serum. Percentages are expressed relative to the samples of bacteria only without antibody or neutrophils. Killing by 1% IgG/IgM-depleted serum in the absence of anti-WTA mAb was 0.3%. The graph presents Mean±SE of 2 experiments counted in quadruplicate.

FIGS. 10A and 10B show the effect of introducing the hexamerization enhancing mutation E430G in IgG1-S4497 on neutrophil-mediated phagocytic uptake of FITC-labeled S. warneri K64 (FIG. 10A) and KV144 (FIG. 10B) bacteria after antibody binding in IgG-depleted NHS. Phagocytic uptake is represented by the MFI (expressed relative to the value for 1.25 μg/mL WT antibody with serum) of gated neutrophils as measured by flow cytometry. Graph A for K64 represents Mean±SE for n=3.

FIGS. 11A-11D show the effect of introducing the hexamerization enhancing mutation E430G in anti-WTA IgG1-6297 on complement binding and deposition and neutrophil-mediated phagocytic uptake of S. aureus. (FIG. 11A) C1q binding after antibody binding to GFP-expressing COL bacteria as determined by flow cytometry. (FIG. 11B) C4b deposition after antibody binding to GFP-expressing COL bacteria as determined by flow cytometry. (FIG. 11C/11D) Neutrophil-mediated phagocytic uptake of GFP-expressing COL (FIG. 11C) and Wood 46 (FIG. 11D) bacteria after antibody binding in IgG-depleted NHS. Phagocytic uptake is represented by the percentage GFP-bacteria containing gated neutrophils (percentage positive neutrophils) as measured by flow cytometry.

### DETAILED DESCRIPTION OF THE INVENTION

In describing the embodiments of the invention specific terminology will be resorted to for the sake of clarity. However, the invention is not intended to be limited to the specific terms so selected, and it is understood that each specific term includes all technical equivalents which operate in a similar manner to accomplish a similar purpose.

As described herein, the inventors of the present invention have found that antibodies binding to WTA or CP such as CP5 and comprising a mutation in the Fc region, was found to be superior at inducing phagocytosis of bacteria expressing WTA or CP such as CP5 compared to the same antibodies with the exception that they do not comprise said mutation in the Fc region. By introducing specific mutations in the Fc region, oligomerization upon target binding on the cell surface can be enhanced, while the antibody molecules remain monomeric in solution WO2013/004842, WO2014/108198.

### Definitions

The term “immunoglobulin” as used herein, refers to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as VH) and a heavy chain constant region. The heavy chain constant region typically is comprised of three domains, CH1, CH2, and CH3. The heavy chains are inter-connected via disulfide bonds in the so-called “hinge region”. Each light chain typically is comprised of a light chain variable region (abbreviated herein as VL) and a light chain constant region. The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901 917 (1987)). Unless otherwise stated or contradicted by context, reference to amino acid positions in the present invention is corresponds to human IgG1 according to EU-numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242). Further unless otherwise stated or contradicted by context, the CDR regions are annotated according to the IMGT definitions.

The term “immunoglobulin IgG”, “IgG” and “immunoglobulin G”, which may be used interchangeably herein refers to an immunoglobulin of the G isotype.

The term “hinge region” as used herein is intended to refer to the hinge region of an immunoglobulin heavy chain. Thus, for example the hinge region of a human IgG1 antibody corresponds to amino acids 216-230 according to the EU numbering.

The term “CH2 region” or “CH2 domain” as used herein is intended to refer the CH2 region of an immunoglobulin heavy chain. Thus, for example the CH2 region of a human IgG1 antibody corresponds to amino acids 231-340 according to the EU numbering. However, the CH2 region may also be any of the other subtypes as described herein.

The term “CH3 region” or “CH3 domain” as used herein is intended to refer to the CH3 region of an immunoglobulin heavy chain. Thus, for example the CH3 region of a human IgG1 antibody corresponds to amino acids 341-447 according to the EU numbering. However, the CH3 region may also be any of the other subtypes as described herein.

The term “fragment crystallizable region”, “Fc region”, “Fc fragment” or “Fc domain”, which may be used interchangeably herein, refers to an antibody region comprising, in the direction from the N- to C-terminal, at least a hinge region, a CH2 domain and a CH3 domain. An Fc region of an IgG1 antibody can, for example, be generated by digestion of an IgG1 antibody with papain. The Fc region of an antibody may mediate the binding of the immunoglobulin to host tissues or factors, including various cells of the immune system (such as effector cells) and components of the complement system such as C1q, the first component in the classical pathway of complement activation.

The term “Fab fragment” in the context of the present invention, refers to a fragment of an immunoglobulin molecule, which comprises the variable regions of the heavy chain and light chain as well as the constant region of the light chain and the CH1 region of an immunoglobulin. The “CH1 region” refers e.g. to the region of a human IgG1 antibody corresponding to amino acids 118-215 according to the EU numbering. Thus, the Fab fragment comprises the binding region of an immunoglobulin.

The term “antibody” (Ab), as used herein to an immunoglobulin molecule, a fragment of an immunoglobulin molecule, or a derivative of either thereof. The antibody of the present invention comprises an Fc-region of an immunoglobulin and an antigen-binding region. An antibody generally contains two CH2-CH3 regions and a connecting region, e.g. a hinge region, e.g. at least an Fc region. Thus the antibody of the present invention may comprise an Fc region and an antigen-binding region. The variable regions of the heavy and light chains of the immunoglobulin molecule contain a binding domain that interacts with an antigen. The term “antibody” as used herein, also refers to unless otherwise specified or contradicted by the context, polyclonal antibodies, monoclonal antibodies (such as human monoclonal antibodies), antibody mixtures (recombinant polyclonal antibodies), chimeric antibodies and humanized antibodies. An antibody of the present invention may be of any isotype.

The term “human antibody”, as used herein, refers to antibodies having variable and constant regions derived from human germline immunoglobulin sequences. The human antibodies of the invention may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “mammal antibody” as used herein, refers to antibodies having variable and constant regions derived from a mammal germline immunoglobulin sequences.

The term “ungulate antibody” as used herein, refers to antibodies having variable and constant regions derived from an ungulate germline immunoglobulin sequences.

The term “chimeric antibody”, as used herein, refers to an antibody in which the heavy chain and the light chain are chimeric as a result of antibody engineering. A chimeric chain is a chain that contains a foreign variable domain (originating from a non-human species, or synthetic or engineered from any species including human) linked to a constant region of human origin.

The term “humanized antibody”, as used herein, refers to an antibody in which the heavy chain and the light chain are humanized as a result of antibody engineering. A humanized chain is typically a chain in which the complementarity determining regions (CDR) of the variable domains are foreign (originating from one species other than human, or synthetic) whereas the remainder of the chain is of human origin. Humanization assessment is based on the resulting amino acid sequence, and not on the methodology per se, which allows protocols other than grafting to be used.

The term “isotype”, as used herein, refers to the immunoglobulin class (for instance IgG1, IgG2, IgG3, IgG4, IgD, IgA1, IgA2, IgE, or IgM) that is encoded by heavy chain constant region genes. To produce a canonical antibody, each heavy chain isotype is to be combined with either a kappa (κ) or lambda (λ) light chain.

The terms “monoclonal antibody”, monoclonal Ab”, “monoclonal antibody composition”, “mAb”, or the like, as used herein refer to a preparation of Ab molecules of single molecular composition. A monoclonal antibody composition displays a single binding specificity and affinity for a particular epitope. Accordingly, the term “human monoclonal antibody” refers to Abs displaying a single binding specificity which have variable and constant regions derived from human germline immunoglobulin sequences. The human mAbs may be generated by a hybridoma which includes a B cell obtained from a transgenic or transchromosomal non-human animal, such as a transgenic mouse, having a genome comprising a human heavy chain transgene repertoire and a human light chain transgene repertoire, rearranged to produce a functional human antibody and fused to an immortalized cell. Further the mAb may also be generated by phage display or other standard methods known to the person skilled in the art.

The term “full-length antibody” when used herein, refers to an antibody (e.g., a parent or variant antibody) which contains all heavy and light chain constant and variable domains corresponding to those that are normally found in a wild-type antibody of that isotype.

The term “oligomer” as used herein, refers to a molecule that consists of more than one but a limited number of monomer units (e.g. antibodies) in contrast to a polymer that, at least in principle, consists of an unlimited number of monomers. Exemplary oligomers are dimers, trimers, tetramers, pentamers and hexamers. Greek prefixes are often used to designate the number of monomer units in the oligomer, for example a tetramer being composed of four units and a hexamer of six units.

The term “oligomerization”, as used herein, is intended to refer to a process that converts molecules to a finite degree of polymerization. Herein, it is observed, that antibodies and/or other dimeric proteins comprising target-binding regions according to the invention can form oligomers, such as hexamers, via non-covalent association of Fc-regions after target binding, e.g., at a cell surface.

The term “Fc-Fc enhancing”, as used herein, is intended to refer to increasing the binding strength between, or stabilizing the interaction between, the Fc regions of two Fc-region containing antibodies or polypeptides so that the polypeptides form oligomers upon target binding.

The term “antigen-binding region”, “antigen binding region”, “binding region” or “antigen binding domain”, as used herein, refers to a region of an antibody which is capable of binding to an antigen. This binding region is typically defined by the VH and VL domains of the antibody which may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). The antigen can be any molecule, such as a polypeptide, e.g. present on a cell, bacterium, or virion. The terms “antigen” and “target” may, unless contradicted by the context, be used interchangeably in the context of the present invention.

The term “target” or “antigen”, as used herein, refers to a molecule to which the antigen binding region of the antibody binds. The target includes any molecule towards which the antibody is directed. The term “antigen” and “target” may in relation to an antibody be used interchangeably and constitute the same meaning and purpose with respect to any aspect or embodiment of the present invention.

The term “binding”, as used herein refers to the interaction of the antigen-binding region of the antibody with the corresponding target. Binding may be determined in a FACS assay as described in Example 5. Antibody binding for the individual antibody is determined as binding above the level of the negative control. As negative control samples without antibody may be used.

The term “epitope” means a protein determinant capable of specific binding to an antibody. Epitopes usually consist of surface groupings of molecules such as amino acids, sugar side chains or a combination thereof and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding (also called immunodominant component of the epitope) and other amino acid residues, which are not directly involved in the binding, such as amino acid residues which are effectively blocked by the specific antigen binding peptide (in other words, the amino acid residue is within the footprint of the specific antigen binding peptide).

As used herein, the term “affinity” refers to the strength of binding of one molecule, e.g. an antibody, to another, e.g. a target or antigen, at a single site, such as the monovalent binding of an individual antigen binding site of an antibody to an antigen.

As used herein, the term “avidity” refers to the combined strength of multiple binding sites between two structures, such as between multiple antigen binding sites of antibodies simultaneously interacting with a target. When more than one binding interaction is present, the two structures will only dissociate when all binding sites dissociate, and thus, the dissociation rate will be slower than for the individual binding sites, and thereby providing a greater effective total binding strength (avidity) compared to the strength of binding of the individual binding sites (affinity).

The term “wall teichoic acid” (WTA) refers to anionic glycopolymers that are covalently linked to the 6-OH group of N-acetylmuramic acid residues in peptidoglycan via a disaccharide consisting of GlcNAc-1-P and N-actelymannosamine followed by two glycerol-phosphate (GroP) units. In one embodiment, the main WTA backbone consists of repeating units of 1,5-d-ribitol-phosphate (RboP) or repeating units of 1,3-l-α-glycerol-phosphate (GroP). In one embodiment, WTA is a ribitol teichoic acid with repeating units of 1,5-phosphodiester linkages of D-ribitol and D-alanyl ester on position 2 and glycosyl substituents on position 4. The glycosyl groups may be N-acetylglucosaminyl α (alpha) or β (beta) as present in S. aureus. The hydroxyls on the alditol/sugar alcohol phosphate repeats are substituted with cationic D-alanine esters and monosaccharides, such as N-acetylglucosamine. In one aspect, the hydroxyl substituents include D-alanyl and alpha (a) or beta (β) GlcNHAc.

The term “antibody binding WTA”, “anti-WTA antibody”, “WTA-binding antibody”, “WTA-specific antibody”, “WTA antibody” may be used interchangeably in the context of the present invention unless contradicted by the context, and refers to any antibody that binds WTA, such as WTA alpha and/or WTA beta. The terms “anti-wall teichoic acid alpha antibody” or “anti-WTA alpha antibody” or “anti-WTAα” or “anti-aGlcNac WTA antibody” are used interchangeably to refer to an antibody that binds wall teichoic acid (WTA) alpha and not WTA beta. Similarly, the terms “anti-wall teichoic acid beta antibody” or “anti-WTA beta antibody” or “anti-WTAβ” or “anti-PGlcNac WTA antibody” are used interchangeably to refer to an antibody that specifically binds wall teichoic acid (WTA) beta. That an antibody binds WTA beta is to be understood as the antibody only binds WTA beta and that the antibody does not cross bind to WTA alpha.

The term “Capsular Polysaccharides” refers to (Capsular polysaccharides are water-soluble, consist of hexosaminuronic acids, and have molecular weights on the order of 100-2000 kDa. They are linear and consist of regularly repeating subunits of one to six monosaccharides) high-molecular-weight capsular polysaccharides that are attached to bacterial cells and surround the bacterial cell surface.

The term “Capsular Polysaccharide type 5”, “CP5” refers to the chemical structure of a Capsular Polysaccharide composed of trisaccharide repeating units of N-acetyl mannosaminuronic acid, N-acetyl L-fucosamine and N-acetyl D-fucosamine (→4)-3-O-Ac-β-D-ManNAcA-(1→4)-α-L-FucNAc-(1→3)-β-D-FucNAc-(1→)ₙ

The terms “antibody binding CP”, “anti-CP antibody”, “CP-binding antibody”, “CP-specific antibody”, and “CP antibody” may be used interchangeably herein and refers to any antibody that binds CP (capsular polysaccharides) on bacteria.

The terms “anti-CP5” and “anti-CP5 antibody” refers to an antibody that binds Capsular Polysaccharide type 5. The term may in particular refer to an antibody that binds CP5 expressed on the Gram-positive bacteria such as S. aureus.

Bacteria are traditionally divided into two main groups, Gram-positive (Gr+) and Gram-negative (Gr−), based upon their Gram-stain retention. Gram-positive bacteria are bounded by a single unit lipid membrane, and they generally contain a thick layer (20-80 nm) of peptidoglycan responsible for retaining the Gram-stain. Gram-positive bacteria are those that are stained dark blue or violet by Gram staining. In contrast, Gram-negative bacteria cannot retain the crystal violet stain, and instead they take up the counterstain (safranin or fuchsine) and appear red or pink in a Gram stain (John G. Holt et al (1994). Bergey's Manual of Determinative Bacteriology (9th ed.). Lippincott Williams & Wilkins. p. 11). Gram-positive cell walls typically lack the outer membrane found in Gram-negative bacteria.

Gram-positive bacteria include but are not limited to the following group of bacterial species, the genera of Staphylococcus, Streptococcus, Bacillus, Clostridium, Corynebacterium, Enterococcus and Listeria.

The term “methicillin-resistant Staphylococcus aureus” (MRSA), alternatively known as multidrug resistant Staphylococcus aureus or oxacillin-resistant Staphylococcus aureus (ORSA), refers to any strain of Staphylococcus aureus that is resistant to beta-lactam antibiotics, which include the penicillins (e.g., methicillin, dicloxacillin, nafcillin, oxacillin, etc.) and the cephalosporins. “Methicillin-sensitive Staphylococcus aureus” (MSSA) refers to any strain of Staphylococcus aureus that is sensitive to beta-lactam antibiotics.

The term “bacteremia” refers to the presence of bacteria in the bloodstream which is most commonly detected through a blood culture. Bacteria can enter the bloodstream as a severe complication of infections (like pneumonia or meningitis), during surgery (especially when involving mucous membranes such as the gastrointestinal tract), or due to catheters and other foreign bodies entering the arteries or veins. Bacteremia can have several consequences. The immune response to the bacteria can cause sepsis and septic shock, which has a relatively high mortality rate. Bacteria can also use the blood to spread to other parts of the body, causing infections at other sites than the original site of infection. Examples of causing infections at other sites than the original site of infection include endocarditis or osteomyelitis.

The term “effector functions” refer to those biological activities attributable to the Fc region of an antibody, which vary with the antibody isotype. Examples of antibody effector functions include: phagocytosis, complement activation, opsonization, phagocyte activation via C5a, phagocyte-dependent bacterial killing C1q-binding, complement activation, complement dependent cytotoxicity (CDC), FcRn binding, Fc-receptor binding including Fc-gamma receptor-binding, Protein A-binding, Protein G-binding, antibody-dependent cellular phagocytosis (ADCP), complement dependent cellular cytotoxicity (CDCC), complement-enhanced cytotoxicity, opsonisation, Fc-containing polypeptide internalization, ADC uptake.

The term “phagocytosis” refers to a process by which a bacteria is engulfed or internalized by a host cell (e.g., macrophage or neutrophil). Phagocytes mediate phagocytosis by three pathways: (i) direct cell surface receptors (for example, lectins, integrins and scavenger receptors), (ii) complement enhanced—using complement receptors (including CR1, receptor for C3b, CR3, CR4, CRIg) to bind and ingest complement opsonized pathogens, and (iii) antibody enhanced—using Fc Receptors (including FcgammaRI, FcgammaRIIA and FcgammaRIIIA) to bind antibody opsonized particles which then become internalized and fuse with lysosomes to become phagolysosomes.

The term “treatment” (and grammatical variations thereof such as “treat” or “treating”) as used herein, refers to clinical intervention designed to alter the natural course of the individual, tissue or cell being treated during the course of clinical pathology. Desirable effects of treatment include, but are not limited to, clearance of the disease causing organism e.g. bacteria, decreasing the rate of disease progression, ameliorating or palliating the disease state, and remission or improved prognosis, all measurable by one skilled in the art such as a physician. In one embodiment, treatment can mean alleviation of symptoms, diminishment of any direct or indirect pathological consequences of the disease, decreasing the rate of infectious disease progression, amelioration or palliation of the disease state, and remission or improved prognosis. In some embodiments, antibodies of the invention are used to delay development of a disease or to slow the progression of an infectious disease.

A “variant” or “antibody variant” of the present invention is an antibody molecule which comprises one or more mutations as compared to a “parent” antibody. Exemplary parent antibodies include, without limitation, a wild-type antibody, a full-length antibody or Fc-containing antibody fragment, a bispecific antibody, a human antibody, humanized antibody, chimeric antibody or any combination thereof.

Exemplary mutations include amino acid deletions, insertions, and substitutions of amino acids in the parent amino acid sequence. Amino acid substitutions may exchange a native amino acid for another naturally-occurring amino acid, or for a non-naturally-occurring amino acid derivative. The amino acid substitution may be conservative or non-conservative. In the context of the present invention, substitutions may be defined by according to the classes of amino acids reflected in one or more of the following three tables:

Amino acid residue classes for conservative substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and Gln (Q) |
| Aliphatic Uncharged Residues | Gly (G), Ala (A), Val (V), Leu (L), and |
| | Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative Physical and Functional Classifications
of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, |
| | and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, P, |
| formation | and T |
| Flexible residues | Q, T, K, S, G, D, E, and R |

For the purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment).

For the purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

The sequence of CDR variants may differ from the sequence of the CDR of the parent antibody sequences through mostly conservative physical or functional amino acids substitutions at most 5 mutations or substitutions selected from conservative, physical or functional amino acids in total across the six CDR sequences of the antibody binding region, such as at most 4 mutations or substitutions selected from conservative, physical or functional amino acids, such as at most 3 mutations or substitutions selected from conservative, physical or functional amino acids, such as at most 2 mutations selected from conservative, physical or functional amino acids or substitutions, such as at most 1 mutation or substitution selected from a conservative, physical or functional amino acid, in total across the six CDR sequences of the antibody binding region. The conservative, physical or functional amino acids are selected from the 20 natural amino acids found i.e, Arg, His, Lys, Asp, Glu, Ser, Thr, Asn, Gln, Cys, Gly, Pro, Ala, Ile, Leu, Met, Phe, Trp, Tyr and Val.

An amino acid or segment in one sequence that “corresponds to” an amino acid or segment in another sequence is one that (i) aligns with the other amino acid or segment using a standard sequence alignment program such as ALIGN, ClustalW or similar.

The term “vector,” as used herein, refers to a nucleic acid molecule capable of inducing transcription of a nucleic acid segment ligated into the vector. One type of vector is a “plasmid”, which is in the form of a circular double stranded DNA loop. Another type of vector is a viral vector, wherein the nucleic acid segment may be ligated into the viral genome. Certain vectors are capable of autonomous replication in a host cell into which they are introduced (for instance bacterial vectors having a bacterial origin of replication and episomal mammalian vectors). Other vectors (such as non-episomal mammalian vectors) may be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present invention is intended to include such other forms of expression vectors, such as viral vectors (such as replication defective retroviruses, adenoviruses and adeno-associated viruses), which serve equivalent functions.

The term “recombinant host cell” (or simply “host cell”), as used herein, is intended to refer to a cell into which an expression vector has been introduced. It should be understood that such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, transfectomas, such as CHO-S cells, HEK-293F cells, Expi293F cells, PER.C6, NS0 cells, and lymphocytic cells, and prokaryotic cells such as E. coli and other eukaryotic hosts such as plant cells and fungi.

### SPECIFIC EMBODIMENTS OF THE INVENTION

The present invention is based, at least in part, on the discovery that the ability of an anti-WTA antibody or anti-CP antibody, such as an anti-CP5 antibody, to induce complement activation resulting in phagocytosis of a bacteria expressing WTA or CP, such as CP5, can be greatly enhanced by introducing a specific mutation in the Fc region corresponding to amino acid position E430, E345 or S440 in human IgG1 according to EU numbering.

The amino acid positions corresponding to E430, E345 and S440 in human IgG1 according to EU numbering are located in the CH3 domain of the Fc region.

By introducing a mutation in the Fc region corresponding to at least one of the positions E430, E345 and S440 in human IgG1 oligomerization upon target binding on the cell surface is enhanced, while the antibody molecules remain monomeric in solution (WO2013/004842; WO2014/108198).

In one embodiment of the present invention the antibody comprises an Fc region wherein a mutation selected from E430G, E345K, E430S, E430F, E430T, E345Q, E345R, E345Y, S440W or S440Y. Thus, in one embodiment of the present invention the antibody comprises an Fc region wherein the mutation is selected from the group consisting of E430G, E345K, E430S, E430F, E430T, E345Q, E345R, E345Y, S440W and S440Y.

In a particular embodiment of the invention the antibody comprises an Fc region wherein the mutation is E430G or E345K.

In one embodiment of the invention the antibody comprises a further substitution in the Fc region corresponding to position K439 or S440, with the proviso that the mutation in S440 is not S440Y or S440W.

Antibodies comprising a Fc-Fc enhancing substitution according to the present invention and a further mutation at position S440 such as S440K do not form oligomers with polypeptides or antibodies comprising a substitution at position S440 such as S440K. Polypeptides or antibodies comprising an Fc-Fc enhancing mutation according to the present invention and a further mutation at position K439 such as K439E do not form oligomers with polypeptides or antibodies comprising a mutation at position K439 such as K439E. In one embodiment of the invention the further mutation is selected from the group consisting of S440K and K439E.

In one embodiment of the present invention the Fc region comprises a further mutation which is a hexamerization-inhibiting such as K439E or S440K. That is in one embodiment of the present invention the Fc region comprises an Fc-Fc enhancing mutation such as E430G and a hexamerization-inhibiting mutation K439E. In one embodiment of the present invention the Fc region comprises a Fc-Fc enhancing mutation such as E345K and a hexamerization-inhibiting mutation such as K439E. In another embodiment of the present invention the Fc region comprises a Fc-Fc enhancing mutation such as E430G and a hexamerization-inhibiting mutation S440K. In one embodiment of the present invention the Fc region comprises an Fc-Fc enhancing mutation such as E345K and hexamerization-inhibiting mutation a S440K. Hereby are embodiments provided that allow for exclusive hexamerization between combinations of antibodies comprising a K439E mutation and antibodies comprising a S440K mutation. That is, the inhibiting mutations K439E and S440K may be viewed as complementary mutations. Combinations of antibodies with two different hexamerization-inhibiting mutations may be of particular interest in compositions having at least two antibodies with different specificities.

In one embodiment of the invention the antibody comprises a) at least one Fc-Fc enhancing mutation at a position selected from the group consisting of: E430, E345 and S440, and b) a K439E or a S440K mutation.

In one aspect the present invention relates to an antibody comprising an Fc region of a human immunoglobulin IgG and an antigen binding region binding to WTA, wherein the Fc region comprises a mutation corresponding to position E430, E345 or S440 in human IgG1 according to EU numbering.

In one embodiment of the present invention the antibody comprises an Fc region of a human immunoglobulin IgG and an antigen binding region binding to WTA on the surface of Gram-positive bacteria, wherein the Fc region comprises a mutation corresponding to position E430, E345 or S440 in human IgG1 according to EU numbering.

In one embodiment of the present invention the antibody comprises an Fc region of a human immunoglobulin IgG and an antigen binding region binding to WTA, wherein the Fc region comprises a mutation corresponding to position E430, E345 or S440 in human IgG1 according to EU numbering In one embodiment of the invention the antigen binding region binds to WTA-alpha. In one embodiment of the invention the antigen binding region binds to WTA-beta.

In one embodiment the anti-WTA antibody comprises an Fc region of human IgG, wherein the Fc region comprises a mutation in an amino acid position corresponding to E430 in human IgG1 according to EU numbering.

In one embodiment the anti-WTA antibody comprises an Fc region of human IgG, wherein the Fc region comprises a mutation selected form the group consisting of: E430G, E345K, E430S, E430F, E430T, E345Q, E345R, E345Y, S440W and S440Y, wherein the mutation corresponds to an amino acid position in human IgG1 according to EU numbering.

In one embodiment the anti-WTA antibody comprises an Fc region of human IgG, wherein the Fc region comprises a mutation selected form the group consisting of: E430G, E430S, E430F and E430T.

In one embodiment the anti-WTA antibody comprise an Fc region of human IgG, wherein the Fc region comprises an E430G mutation.

In one embodiment the anti-WTA antibody comprises an Fc region of human IgG, wherein the Fc region comprises a mutation in an amino acid position corresponding to E345 in human IgG1.

In one embodiment the anti-WTA antibody comprises an Fc region of human IgG, wherein the Fc region comprises a mutation selected form the group consisting of: E345K, E345Q, E345R and E345Y.

In one embodiment the anti-WTA antibody comprise an Fc region of human IgG, wherein the Fc region comprises an E345K mutation.

In one embodiment the anti-WTA antibody comprise an Fc region of human IgG, wherein the Fc region comprises an E345R mutation.

In one embodiment the anti-WTA antibody comprises an Fc region of human IgG, wherein the Fc region comprises a mutation in an amino acid position corresponding to S440 in human IgG1 according to EU numbering.

In one embodiment the anti-WTA antibody comprises an Fc region of human IgG, wherein the Fc region comprises a mutation in an amino acid position corresponding to S440 in human IgG1 according to EU numbering, with the proviso that the mutation in S440 is S440Y or S440W.

In one embodiment the anti-WTA antibody comprises an Fc region of human IgG, wherein the Fc region comprises a mutation selected form the group consisting of: S440Y and S440W.

In one embodiment the anti-WTA antibody comprise an Fc region of human IgG, wherein the Fc region comprises a S440Y mutation.

In one embodiment the anti-WTA antibody comprise an Fc region of human IgG, wherein the Fc region comprises a S440W mutation.

In one embodiment of the present invention the anti-WTA antibody comprises an Fc region of a human immunoglobulin IgG and an antigen binding region binding to WTA, wherein the Fc region comprises an E430G or E345K mutation. In one embodiment the anti-WTA antibody is an anti-WTA-alpha antibody. In another embodiment the anti-WTA antibody is an anti-WTA-beta antibody. That is an antibody according to the present invention comprises an Fc region of a human immunoglobulin G, with a mutation in an amino acid position corresponding to E430, E345 or S440 in human IgG1 according to EU numbering. The anti-WTA antibody according to the invention may be either an anti-WTA-alpha antibody or an anti-WTA-beta antibody.

WTA is expressed on a number of Gram-positive bacteria including Staphylococcus aureus and species in the genera of Staphylococcus, Streptococcus, Bacillus, Clostridium, Corynebacterium, Enterococcus, and Listeria. Thus in one embodiment the WTA is a WTA expressed on one or more of Staphylococcus, Streptococcus, Bacillus, Clostridium, Corynebacterium, Enterococcus, and Listeria. In a further embodiment the WTA is expressed on Staphylococcus aureus. In another embodiment the WTA is expressed on Staphylococcus warneri. WTA can account for as much as 60% of the total cell wall mass in Gram-positive bacteria. The present invention is not limited to particular anti-WTA antibodies and any method for generating an antibody may be used in the context of the present invention. Anti-WTA antibodies may for example may be selected and produced by the methods taught in U.S. Pat. No. 8,283,294; Meijer P J et al (2006) J Mol Biol. 358(3):764-72; Lantto J, et al (2011) J Virol. 85(4): 1820-33.

The chemical structures of WTAs vary among organisms. In S. aureus, WTA is covalently linked to the 6-OH of N-acetyl muramic acid (MurNAc) via a disaccharide composed of N-acetyl glucosamine (GlcNAc)-l-P and N-acetylmannoseamine (ManNAc), which is followed by about two or three units of glycerol-phosphates. The actual WTA polymer is then composed of about 11-40 ribitol-phosphate (Rbo-P) repeating units. The step-wise synthesis of WTA is first initiated by the enzyme called TagO. The repeating units can be further tailored with D-alanine (D-Ala) at C2-OH and/or with N-acetylglucosamine (GlcNAc) at the C4-OH position via a- (alpha) or P-(beta) glycosidic linkages. Depending of the S. aureus strain, or the growth phase of the bacteria the glycosidic linkages could be α-, β-, or a mixture of the two anomers. These GlcNAc sugar modifications are tailored by two specific S. aureus-derived glycosyltransferases (Gtfs): TarM Gtf mediates α-glycosidic linkages, whereas TarS Gtfs mediates β-(beta)glycosidic linkages. The fact that WTA is surface-exposed and consists of multiple repeating epitopes makes it an ideal target for antibody-mediated therapy.

Hereby are provided embodiments of the invention, wherein the antibody of the present invention binds to WTA. In one embodiment of the present invention the antibody comprises an Fc region of a human immunoglobulin IgG and an antigen binding region binding to WTA. In one embodiment of the present invention the antibody comprises an Fc region of a human immunoglobulin IgG and an antigen binding region binding to WTA-alpha. In one embodiment of the present invention the antibody comprises an Fc region of a human immunoglobulin IgG and an antigen binding region binding to WTA-beta. In one embodiment of the present invention the antibody binds WTA-alpha on Gram-positive bacteria. In one embodiment of the present invention the antibody binds WTA-beta on Gram-positive bacteria. In one embodiment of the present invention the antibody binds WTA-alpha on S. aureus. In one embodiment of the present invention the antibody binds WTA-beta on S. aureus.

The antibodies of the invention comprise a mutation in the Fc region that enhances oligomerization when the antibody binds to the bacteria. Without being limited to theory it is believed that the enhanced oligomerization leads to enhanced activation of the complement system and phagocyte-dependent clearance of the bacteria from the host.

Effective eradication of Gram-positive bacteria from the human body largely depends on the phagocytosis of bacteria by professional phagocytes, like neutrophils, that can engulf bacteria and kill them intracellularly. The recurrent infections in patients with neutrophil deficiencies, including many S. aureus infections, show that neutrophils are crucial in human antimicrobial defense against Gram-positive bacteria [Bardoel B W, Kenny E F, Sollberger G, Zychlinsky A: The Balancing Act of Neutrophils. Cell Host Microbe 2014, 15:526-536]. Contact of Gram-positive bacteria with the complement system leads to rapid opsonization of the bacterial surface with C3b/C3bi molecules. This process is essential for phagocytosis of bacteria by phagocytic cells. The antibodies of the invention enhance antibody-dependent complement activation on Gram-positive bacteria and subsequent phagocytosis by immune cells.

In one aspect, the invention provides anti-WTA antibodies which are anti-WTA-α or anti-WTA-β. In one embodiment the anti-WTA antibodies are human monoclonal antibodies. The present invention also encompasses chimeric antibodies and humanized antibodies. In a further embodiment the antibody of the present invention may comprise the CDRs of the present WTA antibodies disclosed in table 1.

In one embodiment of the invention the anti-WTA antibody comprises an antigen binding region comprising a variable heavy chain (VH) region comprising CDR1, CDR2 and CDR3 domains and a variable light chain (VL) region comprising CDR1, CDR2 and CDR3 domains having the amino acid sequences of:

### a) (VH) SEQ ID NO: 1, 2, 3 and (VL) SEQ ID NO: 5, WAS, 6 or

b) the (VH) CDR1, CDR2, CDR3 and (VL) CDR1, CDR2 and CDR3 as defined in a) having one to five mutations or substitutions in total across said six CDR sequences.

That is in one embodiment up to five mutations e.g. substitutions in total are allowed across the six CDRs comprising the antigen binding site. In some embodiments of the invention up to five mutations e.g. substitutions, such as one, two, three, four or five mutations e.g. substitutions, are made across the three CDRs of the VH region and no mutations are made across the CDRs of the VL region. In other embodiments no mutations e.g. substitutions are made across the CDRs of the VH region but up to five mutations e.g. substitutions, such as one, two, three, four or five are found across the CDRs of the VL region.

In one embodiment of the present invention the anti-WTA antibody comprises an Fc region wherein a mutation selected from E430G, E345K, E430S, E430F, E430T, E345Q, E345R, E345Y, S440W or S440Y. In one embodiment of the present invention the anti-WTA antibody comprises an Fc region wherein the mutation is selected from the group consisting of E430G, E345K, E430S, E430F, E430T, E345Q, E345R, E345Y, S440W and S440Y. In a particular embodiment of the invention the anti-WTA antibody comprises an Fc region wherein the mutation is E430G or E345K.

In one embodiment of the invention the anti-WTA antibody comprises an Fc region comprising an E430G or an E345K mutation and an antigen binding region comprising a variable heavy chain (VH) region comprising CDR1, CDR2 and CDR3 domains and a variable light chain (VL) region comprising CDR1, CDR2 and CDR3 domains having the amino acid sequences of:
a) (VH) SEQ ID NO: 1, 2, 3 and (VL) SEQ ID NO: 5, WAS, 6,
b) (VH) SEQ ID NO: 35, 36, 37 and (VL) SEQ ID NO: 39, DAS, 40.

In another aspect of the present invention the antibody comprising an Fc region of a human immunoglobulin IgG and an antigen binding region binding to Capsular polysaccharide (CP) such as Capsular polysaccharide type 5 (CP5) on the surface of bacteria, wherein the Fc region comprises a mutation corresponding to E430, E345 or S440 in human IgG1, EU numbering. In one embodiment the bacteria is a Gram-positive bacteria.

In another embodiment of the present invention the antibody comprising an Fc region of a human immunoglobulin IgG and an antigen binding region binding to Capsular polysaccharide (CP) such as Capsular polysaccharide type 5 (CP5), wherein the Fc region comprises a mutation corresponding to E430, E345 or S440 in human IgG1, EU numbering.

In one embodiment the anti-CP antibody comprises an Fc region of human IgG, wherein the Fc region comprises a mutation in an amino acid position corresponding to E430 in human IgG1 according to EU numbering.

In one embodiment the anti-CP antibody comprises an Fc region of human IgG, wherein the Fc region comprises a mutation selected form the group consisting of: E430G, E345K, E430S, E430F, E430T, E345Q, E345R, E345Y, S440W and S440Y, wherein the mutation corresponds to an amino acid position in human IgG1 according to EU numbering.

In one embodiment the anti-CP antibody comprises an Fc region of human IgG, wherein the Fc region comprises a mutation selected form the group consisting of: E430G, E430S, E430F and E430T.

In one embodiment the anti-CP antibody comprise an Fc region of human IgG, wherein the Fc region comprises an E430G mutation.

In one embodiment the anti-CP antibody comprises an Fc region of human IgG, wherein the Fc region comprises a mutation in an amino acid position corresponding to E345 in human IgG1 according to EU numbering.

In one embodiment the anti-CP antibody comprises an Fc region of human IgG, wherein the Fc region comprises a mutation selected form the group consisting of: E345K, E345Q, E345R and E345Y.

In one embodiment the anti-CP antibody comprise an Fc region of human IgG, wherein the Fc region comprises an E345K mutation.

In one embodiment the anti-CP antibody comprise an Fc region of human IgG, wherein the Fc region comprises an E345R mutation.

In one embodiment the anti-CP antibody comprises an Fc region of human IgG, wherein the Fc region comprises a mutation in an amino acid position corresponding to S440 in human IgG1 according to EU numbering.

In one embodiment the anti-CP antibody comprises an Fc region of human IgG, wherein the Fc region comprises a mutation selected form the group consisting of: S440Y and S440W.

In one embodiment the anti-CP antibody comprise an Fc region of human IgG, wherein the Fc region comprises a S440Y mutation.

In one embodiment the anti-CP antibody comprise an Fc region of human IgG, wherein the Fc region comprises a S440W mutation.

In one embodiment the anti-CP5 antibody comprises an Fc region of human IgG, wherein the Fc region comprises a mutation in an amino acid position corresponding to E430 in human IgG1 according to EU numbering.

In one embodiment the anti-CP5 antibody comprises an Fc region of human IgG, wherein the Fc region comprises a mutation selected form the group consisting of: E430G, E345K, E430S, E430F, E430T, E345Q, E345R, E345Y, S440W and S440Y, wherein the mutation corresponds to an amino acid position in human IgG1 according to EU numbering.

In one embodiment the anti-CP5 antibody comprises an Fc region of human IgG, wherein the Fc region comprises a mutation selected form the group consisting of: E430G, E430S, E430F and E430T.

In one embodiment the anti-CP5 antibody comprise an Fc region of human IgG, wherein the Fc region comprises an E430G mutation.

In one embodiment the anti-CP5 antibody comprises an Fc region of human IgG, wherein the Fc region comprises a mutation in an amino acid position corresponding to E345 in human IgG1 according to EU numbering.

In one embodiment the anti-CP5 antibody comprises an Fc region of human IgG, wherein the Fc region comprises a mutation selected form the group consisting of: E345K, E345Q, E345R and E345Y.

In one embodiment the anti-CP5 antibody comprise an Fc region of human IgG, wherein the Fc region comprises an E345K mutation.

In one embodiment the anti-CP5 antibody comprise an Fc region of human IgG, wherein the Fc region comprises an E345R mutation.

In one embodiment the anti-CP5 antibody comprises an Fc region of human IgG, wherein the Fc region comprises a mutation in an amino acid position corresponding to S440 in human IgG1 according to EU numbering.

In one embodiment the anti-CP5 antibody comprises an Fc region of human IgG, wherein the Fc region comprises a mutation selected form the group consisting of: S440Y and S440W.

In one embodiment the anti-CP5 antibody comprise an Fc region of human IgG, wherein the Fc region comprises a S440Y mutation.

In one embodiment the anti-WTA antibody comprise an Fc region of human IgG, wherein the Fc region comprises a S440W mutation.

Capsular polysaccharides are common virulence structures of pathogenic bacteria causing invasive disease. Capsules increase bacterial virulence by rendering the bacterium resistant to phagocytosis. Capsular polysaccharide type 5 (CP5) is the main serotypes produced by clinical S. aureus strains are the serotype consisting of capsular polysaccharide 5 (CP5), accounting for ^{˜}75% of all clinical isolates. The expression of CP5 has been shown to enhance virulence and survival of S. aureus in vivo. Next to inhibition of phagocytic uptake, CP5 expression has been described to provide protection against intracellular killing of the bacterium. S. aureus produces various surface polysaccharides and most strains express capsular polysaccharides (CPs) in vivo or under defined culture conditions. Phagocytosis and killing by neutrophil granulocytes play a key role in defense against S. aureus infections. Most CPs have been shown to have antiphagocytic properties. The fact that CP is surface-exposed and consists of multiple repeating epitopes makes it an ideal target for antibody-mediated therapy.

CP5 expressed on S. aureus is composed of 2-acetamido-2-deoxy-L-fucose (1 part), 2-acetamido-2-deoxy-D-fucose (1 part), and 2-acetamido-2-deoxy-D-mannuronic acid (1 part).

In one embodiment of the invention the anti-CP5 antibody comprises an antigen binding region comprising a variable heavy chain (VH) region comprising CDR1, CDR2 and CDR3 domains and a variable light chain (VL) region comprising CDR1, CDR2 and CDR3 domains having the amino acid sequences of:

### a) (VH) SEQ ID NO: 8, 9, 10 and (VL) SEQ ID NO: 12, LAS, 13; or

b) the (VH) CDR1, CDR2, CDR3 and (VL) CDR1, CDR2 and CDR3 as defined in a) having one to five mutations or substitutions in total across said six CDR sequences.

That is in one embodiment up to five mutations e.g. substitutions in total are allowed across the six CDRs comprising the antigen binding site. In some embodiments of the invention up to five mutations e.g. substitutions such as one, two, three, four or five mutations e.g. substitutions, are made across the three CDRs of the VH region and no mutations are made across the CDRs of the VL region. In other embodiments no mutations e.g. substitutions are made across the CDRs of the VH region but up to five mutations e.g. substitutions, such as one, two, three, four or five are found across the CDRs of the VL region.

In one embodiment of the present invention the anti-CP5 antibody comprises an Fc region wherein a mutation selected from E430G, E345K, E430S, E430F, E430T, E345Q, E345R, E345Y, S440W or S440Y. In one embodiment of the present invention the anti-CP5 antibody comprises an Fc region wherein the mutation is selected from the group consisting of E430G, E345K, E430S, E430F, E430T, E345Q, E345R, E345Y, S440W and S440Y.

In a particular embodiment of the invention the anti-CP5 antibody comprises an Fc region wherein the mutation is E430G or E345K.

The antibodies of the invention are useful as antimicrobial agents effective against a number of human and veterinary Gram-positive bacteria, including the genera of Staphylococci, for example S. aureus, S. epidermidis, S. saprophyticus, S. simulans and S. warneri; Listeria, for example L. monocytogenes; Enterococci, for example E. faecalis, E. faecium; Streptococci, for example S. pneumoniae, S. pyogenes, S. agalactiae, S. suis; Bacillus, for example B. anthracis, Clostridium, for example C. difficile; Corynebacterium, for example C. diphteriae.

Following entry into the bloodstream, S. aureus can cause metastatic infection in almost any organ. Secondary infections occur in about one-third of cases before the start of therapy (Fowler et al, (2003) Arch. Intern. Med. 163:2066-2072), and even in 10% of patients after the start of therapy (Khatib et al., (2006) Scand. J. Infect. Dis., 38:7-14). Hallmarks of infections are large reservoirs of pus, tissue destruction, and the formation of abcesses (all of which contain large quantities of neutrophils). While only about 5% of patients develop complications if the bacteremia is extinguished within 48 hours, the levels rises to 40%, if bacteraemia persists beyond three days. Staphylococcus aureus is the leading cause of surgical site infections (SSI). In particular, SSI caused by methicillin-resistant Staphylococcus aureus (MRSA) has emerged as a devastating complication, leading to increased mortality rates, increased length of hospitalization, and increased costs. It is a leading cause of bacteremia and infective endocarditis as well as osteoarticular, skin and soft tissue, pleuropulmonary, and device-related infections.

In one embodiment of the invention the Gram-positive bacteria is selected from the following group: species in the genera of Staphylococcus, Streptococcus, Bacillus, Clostridium, Corynebacterium, Enterococcus, and Listeria.

In one embodiment of the invention the Staphylococcus is e.g. S. aureus, S. saprophyticus, S. warneri; or S. simulans. In one embodiment of the invention the Streptococcus is e.g. S. pneumoniae. In one embodiment of the invention the Clostridium is e.g. C. difficile. In one embodiment the Enterococcus is e.g E. faecalis. In one embodiment of the invention the Listeria is e.g. Listeria monocytogenes.

In a particular embodiment of the invention the antibody binds to WTA or CP5 on Gram-positive bacteria that is S. aureus. In another embodiment of the invention the Staphylococcus aureus (S. aureus) is methicillin-resistant S. aureus (MRSA) or methicillin-sensitive S. aureus (MSSA). In a further embodiment of the invention the S. aureus is resistant or insensitive to previous treatment with a drug. That is the S. aureus may be resistant to previous treatment with an antibiotic such as trimethoprim-sulfametoxazole (TMP-SMX), clindamycin, doxycycline, minocycline, tetracycline, rifampin, vancomycin or linezolid.

In one embodiment of the present invention the antibody is a monoclonal antibody. In one embodiment of the present invention the antibody is an IgG1, IgG2, IgG3, IgG4, IgE, IgD or IgM isotype. In a preferred embodiment of the invention the antibody is an IgG1 or IgG2 isotype.

In one embodiment of the invention the antibody is a mammal, human or ungulate antibody.

In one embodiment of the invention the antibody is a humanized or chimeric antibody.

In one embodiment of the invention the antibody is a monoclonal antibody.

In one embodiment of the invention the light chain is a kappa or a lambda.

In one embodiment the antibody is a full length antibody.

In one embodiment of the present invention the antibody comprises an Fc region comprising an amino acid sequence of the following group:
a) SEQ ID NO 15: IgG1, 430G;
b) SEQ ID NO 16: IgG1, 345K;
c) SEQ ID NO 17: IgG1, 430S;
d) SEQ ID NO 18: IgG1, E430F;
e) SEQ ID NO 19: IgG1, E430T;
f) SEQ ID NO 20: IgG1, E345Q;
g) SEQ ID NO 21: IgG1, E345R;
h) SEQ ID NO 22: IgG1, E345Y;
i) SEQ ID NO 23: IgG1, S440W;
j) SEQ ID NO 24: IgG1, S440 or
   k) any of a) to j) having one to five mutations or substitutions in total across said sequence. That is in one embodiment up to five mutations or substitutions in total are allowed across the Fc region. In some embodiments of the invention up to five mutations or substitutions such as one, two, three, four or five mutations or substitutions, are allowed across the Fc region. Hereby are embodiments provided that allow for conservative mutations in the Fc region, such mutations may not impair the hexamer enhancing function of the Fc region according to the invention.

In one embodiment of the present invention the antibody comprises an Fc region comprising an amino acid sequence of the following group:
a) SEQ ID NO 25: IgG2, E430G;
b) SEQ ID NO 26: IgG2, E345K;
c) SEQ ID NO 27: IgG2, E430S;
d) SEQ ID NO 28: IgG2, E430F;
e) SEQ ID NO 29: IgG2, E430T;
f) SEQ ID NO 30: IgG2, E345Q;
g) SEQ ID NO 31: IgG2, E345R;
h) SEQ ID NO 32: IgG2, E345Y;
i) SEQ ID NO 33: IgG2, S440W;
j) SEQ ID NO 34: IgG2, S440 or
   k) any of a) to j) having one to five mutations or substitutions in total across said sequence. That is in one embodiment up to five mutations or substitutions in total are allowed across the Fc region. In some embodiments of the invention up to five mutations or substitutions such as one, two, three, four or five mutations or substitutions, are allowed across the Fc region. Hereby are embodiments provided that allow for conservative mutations in the Fc region, such mutations may not impair the hexamer enhancing function of the Fc region according to the invention.

In one embodiment of the present invention the antibody comprises an Fc region comprising an amino acid sequence of the following group consisting of:
a) SEQ ID NO 15: IgG1, 430G;
b) SEQ ID NO 16: IgG1, 345K;
c) SEQ ID NO 17: IgG1, 430S;
d) SEQ ID NO 18: IgG1, E430F;
e) SEQ ID NO 19: IgG1, E430T;
f) SEQ ID NO 20: IgG1, E345Q;
g) SEQ ID NO 21: IgG1, E345R;
h) SEQ ID NO 22: IgG1, E345Y;
i) SEQ ID NO 23: IgG1, S440W and
j) SEQ ID NO 24: IgG1, S440
wherein the Fc region of a) to j) further comprises a K439E or S440K substitution.

In one embodiment of the present invention the antibody comprises an Fc region comprising an amino acid sequence of the following group consisting of:
a) SEQ ID NO 25: IgG2, E430G;
b) SEQ ID NO 26: IgG2, E345K;
c) SEQ ID NO 27: IgG2, E430S;
d) SEQ ID NO 28: IgG2, E430F;
e) SEQ ID NO 29: IgG2, E430T;
f) SEQ ID NO 30: IgG2, E345Q;
g) SEQ ID NO 31: IgG2, E345R;
h) SEQ ID NO 32: IgG2, E345Y;
i) SEQ ID NO 33: IgG2, S440W and
j) SEQ ID NO 34: IgG2, S440;
wherein the Fc region of a) to j) further comprises a K439E or S440K substitution.

In one embodiment of the invention the antibody enhances phagocytosis. Without being limited to theory it is believed that when the antibodies of the invention bind to WTA or CP5 on bacteria the mutation in the Fc region enhances oligomerization of the antibodies on the bacteria. The formation of oligomeric antibody structures such as hexametric structures on bacteria enhances phagocytosis of the bacteria by immune cells, such as neutrophils, macrophages and dendritic cells. The antibodies of the invention enhance antibody-dependent complement activation on Gram-positive bacteria and subsequent phagocytosis by immune cells.

In one embodiment of the present invention the anti-WTA antibody induces oligomerization of antibodies on target cells expressing WTA, such anti-WTA antibodies may be either anti-WTA-α antibodies or anti-WTA-β antibodies binding to either WTA-α or WTA-β on Gram-positive bacteria.

In one embodiment of the present invention the anti-CP5 antibody induces oligomerization, such as hexamerization of antibodies on target cells expressing CP5.

In one embodiment of the invention the antibody is enhancing phagocytosis in the presence of complement. That is oligomerization of the anti-WTA or anti-CP5 antibodies on the bacteria enhances binding of the complement factor C1q to the Fc region of the antibody creating a C1q:antibody complex, which allows binding of C1q to C1q receptors on phagocytic cells thereby enhancing phagocytosis.

In one embodiment of the invention the antibody enhances phagocytosis of bacteria by immune cells. That is antibodies of the invention may enhance phagocytosis by immune cells such as neutrophils, monocytes, macrophages, kupffer cells, dendritic cells, antigen-presenting cells.

In one embodiment of the invention the antibody is enhancing neutrophil-mediated phagocytosis. Neutrophil-mediated phagocytosis may be determined as in example 6 or example 8. An antibody according the invention is incubated with human serum, fluorescently labeled S. aureus and human neutrophils. Phagocytosis is quantified by flow cytometry.

In one embodiment of the invention the antibody enhances complement activation on Gram-positive bacteria. That is in one embodiment the antibody enhances activation of complement protein C4 into C4b. In one embodiment the antibody enhances activation of complement protein C3 into C3b. Activation of C3 on the Gram-positive bacterial cells leads to opsonization of the bacteria by C3-derived opsonins (C3b and C3bi)

In one embodiment of the invention the antibody is enhancing complement-mediated phagocyte activation. That is in one embodiment the antibody enhances formation of the chemoattractant C5a.

In one embodiment of the invention the antibody is enhancing complement-mediated killing.

### Compositions

One aspect of the invention relates to a composition comprising an antibody according to the present invention. Thus a composition according to the present invention comprises an antibody according to any embodiment described herein.

The anti-WTA antibodies, anti-CP antibodies or anti-CP5 antibodies such as monoclonal antibodies according to any aspect or embodiment of the present invention may be comprised in a composition, such as a pharmaceutical composition, diagnostic composition or any other composition.

In one embodiment the present invention relates to a composition comprising an antibody according to the present invention and a pharmaceutical carrier or a pharmaceutical excipient.

In one aspect the present invention relates to a composition comprising an antibody according to any aspect described herein.

In one aspect the present invention relates to a composition comprising an antibody with an antigen binding region binding to WTA or CP such as CP5, wherein the Fc region comprises a mutation corresponding to amino acid position E430, E345 or S440 in human IgG1 according to EU numbering.

In one embodiment of the present invention the composition comprises an antibody with an antigen binding region binding to WTA, wherein the Fc region comprises a mutation selected from the group consisting of E430G, E345K, E430S, E430F, E430T, E345Q, E345R, E345Y, S440W and S440Y. In one embodiment the composition comprises an antibody binding to WTA-alpha. In one embodiment the composition comprises an antibody binding to WTA-beta.

In one embodiment of the invention the composition comprises an anti-WTA antibody comprising an Fc region of human IgG, wherein the Fc region comprises a mutation in an amino acid position corresponding to E430 in human IgG1.

In one embodiment of the invention the composition comprises an anti-WTA antibody comprising an Fc region of human IgG, wherein the Fc region comprises a mutation selected form the group consisting of: E430G, E430S, E430F and E430T.

In one embodiment of the invention the composition comprises an anti-WTA antibody comprising an Fc region of human IgG, wherein the Fc region comprises an E430G mutation.

In one embodiment of the invention the composition comprises an anti-WTA antibody comprising an Fc region of human IgG, wherein the Fc region comprises a mutation in an amino acid position corresponding to E345 in human IgG1.

In one embodiment of the invention the composition comprises an anti-WTA antibody comprising an Fc region of human IgG, wherein the Fc region comprises a mutation selected form the group consisting of: E345K, E345Q, E345R and E345Y.

In one embodiment of the invention the composition comprises an anti-WTA antibody comprising an Fc region of human IgG, wherein the Fc region comprises an E345K mutation.

In one embodiment of the invention the composition comprises an anti-WTA antibody comprising an Fc region of human IgG, wherein the Fc region comprises an E345R mutation.

In one embodiment of the invention the composition comprises an anti-WTA antibody comprising an Fc region of human IgG, wherein the Fc region comprises a mutation in an amino acid position corresponding to S440 in human IgG1.

In one embodiment of the invention the composition comprises an anti-WTA antibody comprising an Fc region of human IgG, wherein the Fc region comprises a mutation selected form the group consisting of: S440Y and S440W.

In one embodiment of the invention the composition comprises an anti-WTA antibody comprising an Fc region of human IgG, wherein the Fc region comprises a S440Y mutation.

In one embodiment of the invention the composition comprises an anti-WTA antibody comprising an Fc region of human IgG, wherein the Fc region comprises a S440W mutation.

In one embodiment of the present invention the composition comprises an anti-WTA, antibody comprising an Fc region of a human immunoglobulin IgG and an antigen binding region binding to WTA, wherein the Fc region comprises a mutation corresponding to E430G or E345K in human IgG1 according to EU numbering. In one embodiment of the invention the composition comprises an anti-WTA antibody which is an anti-WTA-α antibody. In another embodiment of the invention the composition comprises an anti-WTA antibody which is an anti-WTA-β antibody. That is an antibody according to the present invention comprises an Fc region of a human immunoglobulin G, with a mutation corresponding to amino acid position E430, E345 or S440 in human IgG1 according to EU numbering. The anti-WTA antibody according to the invention may be either an anti-WTA-α antibody or an anti-WTA-β antibody.

In one embodiment of the present invention the composition comprise an antibody with an antigen binding region binding to CP, wherein the Fc region comprises a mutation selected from the group consisting of E430G, E345K, E430S, E430F, E430T, E345Q, E345R, E345Y, S440W and S440Y.

In one embodiment of the invention the composition comprises an anti-CP antibody comprising an Fc region of human IgG, wherein the Fc region comprises a mutation selected form the group consisting of: E430G, E430S, E430F and E430T.

In one embodiment of the invention the composition comprises an anti-CP antibody comprising an Fc region of human IgG, wherein the Fc region comprises an E430G mutation.

In one embodiment of the invention the composition comprises an anti-CP antibody comprising an Fc region of human IgG, wherein the Fc region comprises a mutation in an amino acid position corresponding to E345 in human IgG1.

In one embodiment of the invention the composition comprises an anti-CP antibody comprising an Fc region of human IgG, wherein the Fc region comprises a mutation selected form the group consisting of: E345K, E345Q, E345R and E345Y.

In one embodiment of the invention the composition comprises an anti-CP antibody comprising an Fc region of human IgG, wherein the Fc region comprises an E345K mutation.

In one embodiment of the invention the composition comprises an anti-CP antibody comprising an Fc region of human IgG, wherein the Fc region comprises an E345R mutation.

In one embodiment of the invention the composition comprises an anti-CP antibody comprising an Fc region of human IgG, wherein the Fc region comprises a mutation in an amino acid position corresponding to S440 in human IgG1.

In one embodiment of the invention the composition comprises an anti-CP antibody comprising an Fc region of human IgG, wherein the Fc region comprises a mutation selected form the group consisting of: S440Y and S440W.

In one embodiment of the invention the composition comprises an anti-CP antibody comprising an Fc region of human IgG, wherein the Fc region comprises a S440Y mutation.

In one embodiment of the invention the composition comprises an anti-CP antibody comprising an Fc region of human IgG, wherein the Fc region comprises a S440W mutation.

In one embodiment of the present invention the composition comprises an anti-CP, antibody comprising an Fc region of a human immunoglobulin IgG and an antigen binding region binding to CP, wherein the Fc region comprises a mutation corresponding to E430G or E345K in human IgG1 according to EU numbering. In one embodiment of the invention the anti-CP antibody according to the present invention comprises an Fc region of a human immunoglobulin G, with a mutation corresponding to amino acid position E430, E345 or S440 in human IgG1 according to EU numbering.

In one embodiment of the present invention the composition comprise an antibody with an antigen binding region binding to CP5, wherein the Fc region comprises a mutation selected from the group consisting of E430G, E345K, E430S, E430F, E430T, E345Q, E345R, E345Y, S440W and S440Y.

In one embodiment of the invention the composition comprises an anti-CP5 antibody comprising an Fc region of human IgG, wherein the Fc region comprises a mutation selected form the group consisting of: E430G, E430S, E430F and E430T.

In one embodiment of the invention the composition comprises an anti-CP5 antibody comprising an Fc region of human IgG, wherein the Fc region comprises an E430G mutation.

In one embodiment of the invention the composition comprises an anti-CP5 antibody comprising an Fc region of human IgG, wherein the Fc region comprises a mutation in an amino acid position corresponding to E345 in human IgG1.

In one embodiment of the invention the composition comprises an anti-CP5 antibody comprising an Fc region of human IgG, wherein the Fc region comprises a mutation selected form the group consisting of: E345K, E345Q, E345R and E345Y.

In one embodiment of the invention the composition comprises an anti-CP5 antibody comprising an Fc region of human IgG, wherein the Fc region comprises an E345K mutation.

In one embodiment of the invention the composition comprises an anti-CP5 antibody comprising an Fc region of human IgG, wherein the Fc region comprises an E345R mutation.

In one embodiment of the invention the composition comprises an anti-CP5 antibody comprising an Fc region of human IgG, wherein the Fc region comprises a mutation in an amino acid position corresponding to S440 in human IgG1.

In one embodiment of the invention the composition comprises an anti-CP5 antibody comprising an Fc region of human IgG, wherein the Fc region comprises a mutation selected form the group consisting of: S440Y and S440W.

In one embodiment of the invention the composition comprises an anti-CP5 antibody comprising an Fc region of human IgG, wherein the Fc region comprises a S440Y mutation.

In one embodiment of the invention the composition comprises an anti-CP5 antibody comprising an Fc region of human IgG, wherein the Fc region comprises a S440W mutation.

In one embodiment of the present invention the composition comprises an anti-CP5, antibody comprising an Fc region of a human immunoglobulin IgG and an antigen binding region binding to CP5, wherein the Fc region comprises a mutation corresponding to E430G or E345K in human IgG1 according to EU numbering. In one embodiment of the invention the anti-CP5 antibody according to the present invention comprises an Fc region of a human immunoglobulin G, with a mutation corresponding to amino acid position E430, E345 or S440 in human IgG1 according to EU numbering.

In one embodiment of the invention the composition comprises an antibody wherein the antibody comprises a further hexamerization-inhibiting mutation such as K439E or S440K. That is in one embodiment of the present invention the Fc region comprises an Fc-Fc enhancing mutation and a hexamerization-inhibiting mutation selected from the group consisting of K439E and S440K.

Antibodies comprising an Fc-Fc enhancing mutation at a position selected form the group consisting of E430 and E345 and a further comprising a S440K mutation do not form oligomers with antibodies comprising a S440K mutation. Antibodies comprising an Fc-Fc enhancing mutation at a position selected form the group consisting of E430 and E345 and a further comprising a K439E mutation do not form oligomers with antibodies comprising a K439E mutation. In one embodiment the composition comprises a first antibody and a second antibody, wherein the first and second antibody comprises a mutation at a position selected from the group consisting of E430 and E345, wherein the first antibody further comprises a K439E mutation and the second antibody further comprises a S440K mutation. In one embodiment the composition comprises a first antibody and a second antibody, wherein the first and second antibody comprises a mutation at a position selected from the group consisting of E430 and E345, wherein the first antibody further comprises a S440K mutation and the second antibody further comprises a K439E mutation.

In one embodiment of the present invention the composition comprises a first antibody and a second antibody, wherein the first and second antibody comprises a mutation at a position selected from the group consisting of E430 and E345, wherein the first antibody further comprises a S440K mutation and the second antibody further comprises a K439E mutation.

In one embodiment of the present invention the composition comprises a first antibody and a second antibody, wherein the first or second antibody comprises a S440W or a S440Y mutation and wherein the first antibody and or second antibody further comprises an K439E mutation.

In one embodiment of the present invention the composition comprises an antibody comprising an Fc region comprises a Fc-Fc enhancing mutation such as E430G and a hexamerization-inhibiting mutation K439E. In one embodiment of the present invention the composition comprises an antibody comprises an Fc region comprising an Fc-Fc enhancing mutation such as E345K and a hexamerization-inhibiting mutation K439E. In another embodiment of the present invention the composition comprises an antibody comprising an Fc region comprising an Fc-Fc enhancing mutation such as E430G and a hexamerization-inhibiting mutation S440K. In one embodiment of the present invention the composition comprises an antibody comprising an Fc region comprises a Fc-Fc enhancing mutation such as E345K and hexamerization-inhibiting such as S440K. Hereby are embodiments provided that allow for exclusive hexamerization between combinations of antibodies comprising a K439E mutation and antibodies comprising a S440K mutation. That is, the inhibiting mutations K439E and S440K may be viewed as complementary mutations. Combinations of antibodies with two different hexamerization-inhibiting substitutions may be of particular interest in compositions having at least two antibodies with different specificities.

In one embodiment of the invention the composition comprises at least one antibody comprising a) at least one Fc-Fc enhancing substitution at a position selected from the group consisting of: E430, E345 and S440, and b) a K439E mutation.

In one embodiment of the invention the composition comprises at least one antibody comprising a) at least one Fc-Fc enhancing mutation at a position selected from the group consisting of: E430 and E345, and b) a S440K mutation.

In one embodiment of the invention the composition comprises an antibody wherein the antibody comprises a further hexamerization-inhibiting mutation corresponding to K439E or S440K in human IgG1, according to EU numbering. That is in one embodiment of the present invention the Fc region comprises an Fc-Fc enhancing mutation and a hexamerization-inhibiting mutation. Thus in one embodiment the Fc region comprises an Fc-Fc enhancing mutation, such as E430G or E345K, and hexamerization-inhibiting mutation, such as K439E. In one embodiment of the present invention the Fc region comprise an E430G mutation and a K439E mutation. In one embodiment of the present invention the Fc region comprise an E345K mutation and a K439E mutation. In another embodiment of the present invention the Fc region comprise an Fc-Fc enhancing mutation such as E430G or E345K, and hexamerization-inhibiting mutation, such as S440K. In one embodiment of the present invention the Fc region comprise an E430G mutation and a S440K mutation. In one embodiment of the present invention the Fc region comprise an E345K mutation and a S440K mutation.

In one embodiment of the invention the composition comprises a first antibody comprising a mutation selected from the group consisting of: E430G, E345K, E430S, E430F, E430T, E345Q, E345R and E345Y; and a second antibody comprising a mutation selected from the group consisting of E430G, E345K, E430S, E430F, E430T, E345Q, E345R, E345Y, S440W and S440Y, wherein the first antibody further comprises a K439E mutation and the second antibody further comprises a S440K mutation.

In one embodiment of the invention the composition comprises a first antibody comprising a mutation selected from the group consisting of: E430G, E345K, E430S, E430F, E430T, E345Q, E345R, E345Y, S440W and S440Y; and a second antibody comprising a mutation selected from the group consisting of E430G, E345K, E430S, E430F, E430T, E345Q, E345 and, E345Y, wherein the first antibody further comprises a K439E mutation and the second antibody further comprises a S440K mutation. Hereby are embodiments provided that allow for exclusive hexamerization between combinations of antibodies comprising a K439E mutation and antibodies comprising a S440K mutation. Without being bound by theory a combination of a first and a second antibody comprising a K439K and S440K mutation, respectively, is believed to allow for oligomerization of the first and second antibody upon target binding on the cell surface of bacteria.

One embodiment of the invention relates to a composition comprising at least one anti-WTA or an anti-CP such as an anti-CP5 antibody according to any one of the embodiments described herein.

An additional embodiment of the invention relates to a composition comprising two or more anti-WTA or two or more anti-CP antibodies, such as anti-CP5 antibodies, according to any one of the embodiments described herein. The composition may comprise, one, two or more anti-WTA antibodies or one, two or more anti-CP5 antibodies according to the invention as described herein that are not identical, such as a combination of two different anti-WTA antibodies or two different anti-CP5 antibodies or a combination of one or more anti-WTA and one or more anti-CP5 antibodies. In one embodiment of the present invention the composition comprises a combination of two or more anti-WTA antibodies binding to different WTA molecules, such as WTA-alpha and WTA-beta, or to different epitopes on the same WTA molecule. In one embodiment of the present invention the composition comprises a combination of two or more anti-CP5 antibodies binding different epitopes on CP5.

In one embodiment the composition may comprise one or more anti-WTA antibodies or, anti-CP antibodies, such as anti-CP5 antibodies, in combination with other antibodies. In one embodiment the composition may comprise polyclonal antibodies, wherein one or more anti-WTA antibodies or one or more anti-CP antibodies such as one or more anti-CP5 antibodies according to the present invention are included in the composition.

In one embodiment the composition of the present invention may comprise an anti-WTA antibody according to the present invention and an anti-CP, such as an anti-CP5, antibody according to the present invention.

In a further embodiment such a composition comprising an anti-WTA and an anti-CP, such as anti-CP5 antibody may comprise any combination of anti-WTA antibodies and/or any combination of anti-CP, such as anti-CP5, antibodies described herein.

In one embodiment of the invention the composition comprises an antibody in a pharmaceutical composition. That is the composition may comprise an anti-WTA antibody or an anti-CP5 antibody according to the present invention in a pharmaceutical composition. Thus the composition may comprise a pharmaceutical carrier or a pharmaceutical excipient.

In a further embodiment the composition of the present invention may comprise a pharmaceutical carrier or pharmaceutical excipient.

The antibodies or compositions according to any aspect or embodiment of the present invention may be used as a medicament, i.e. for therapeutic applications.

In one embodiment of the present invention the composition comprises one or more antibodies according to the invention such as monoclonal antibodies for use as a medicament.

In one embodiment of the invention the antibody or composition is for use in treatment of an infection caused by a bacteria. In one embodiment of the invention the antibody or composition is for use in treatment of Gram-positive bacteria. The Gram positive bacteria may be selected from the following group of Staphylococcus, Streptococcus, Bacillus, Clostridium, Corynebacterium, Enterococcus, and Listeria.

In one embodiment of the invention the Staphylococcus is e.g. S. aureus, S. saprophyticus, S. warneri; or S. simulans. In one embodiment of the invention the Streptococcus is e.g. S. pneumoniae. In one embodiment of the invention the Clostridium is e.g. C. difficile. In one embodiment the Enterococcus is e.g E. faecalis. In one embodiment of the invention the Listeria is e.g. Listeria monocytogenes.

In a particular embodiment of the invention the antibody or composition is for use in treatment of an infection caused by Staphylococcus aureus, MRSA or MSSA.

In one embodiment of the invention the antibody or composition is for use in preventive treatment of an infection caused by Gram-positive bacteria. In on embodiment of the invention the antibody or composition is for use in prophylaxis treatment of an infection caused by Gram-positive bacteria.

S. aureus, MSSA and MRSA may cause the following one or more of the following diseases: Surgical Site Infections (SSI), wound infections, cystic fibrosis, pneumonia, ventilator-associated pneumonia (yap), sepsis, toxic shock syndrome, Intravenous line infections and infections in the presence of prosthetic devices.

In one embodiment of the invention the antibody or composition is for use in treatment of a disease selected form the group of: Surgical Site Infections (SSI), wound infections, cystic fibrosis, pneumonia, ventilator-associated pneumonia (yap), sepsis, toxic shock syndrome, Intravenous line infections and infections in the presence of prosthetic devices.

In one embodiment of the invention the antibody or composition is for use in treatment of meningitis, urinary tract infections or pneumoniae.

In one embodiment of the invention the composition comprises a pharmaceutical excipient.

In one embodiment of the invention the composition comprises one or more pharmaceutical excipients.

In one embodiment of the invention the antibody or composition is a pharmaceutical composition.

In one embodiment of the invention the antibody is comprised in a pharmaceutical composition.

Pharmaceutical compositions of the present invention may comprise antibodies such as monoclonal antibodies according to any aspect or embodiment of the present invention.

The pharmaceutical composition of the present invention may contain one or more antibodies, such as monoclonal antibodies, of the present invention, a combination of an antibody according to the invention with another therapeutic compound, or a combination of compounds of the present invention.

In one embodiment of the invention the antibody of the present invention is comprised in a pharmaceutical composition.

Pharmaceutical compositions of the present invention may comprise antibodies according to any aspect or embodiment of the present invention.

The pharmaceutical compositions may be formulated with pharmaceutically acceptable carriers or diluents as well as any other known adjuvants and excipients in accordance with conventional techniques such as those disclosed in (Rowe et al., Handbook of Pharmaceutical Excipients, 2012 June, ISBN 9780857110275)

The pharmaceutically acceptable carriers or diluents as well as any other known adjuvants and excipients should be suitable for the antibody of the present invention and the chosen mode of administration. Suitability for carriers and other components of pharmaceutical compositions is determined based on the lack of significant negative impact on the desired biological properties of the chosen compound or pharmaceutical composition of the present invention (e.g., less than a substantial impact (10% or less relative inhibition, 5% or less relative inhibition, etc.) upon antigen binding).

A pharmaceutical composition of the present invention may also include diluents, fillers, salts, buffers, detergents, stabilizers, preservatives, tissue fixatives, solubilizers, and/or other materials suitable for inclusion in a pharmaceutical composition.

The actual dosage levels of the active ingredients i.e. antibody in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The pharmaceutical composition may be administered by any suitable route and mode. Suitable routes of administering a compound of the present invention in vivo and in vitro are well known in the art and may be selected by those of ordinary skill in the art.

In one embodiment, the pharmaceutical composition of the present invention is administered parenterally.

The terms “parenteral administration” and “administered parenterally” as used herein refers to modes of administration other than enteral and topical administration, usually by injection, and include epidermal, intravenous, intramuscular, intra-arterial, intrathecal, intracapsular, intra-orbital, intracardiac, intradermal, intraperitoneal, intratendinous, transtracheal, subcutaneous, subcuticular, intra-articular, subcapsular, subarachnoid, intraspinal, intracranial, intrathoracic, epidural and intrasternal injection and infusion.

In one embodiment, the pharmaceutical composition of the present invention is administered by intravenous or subcutaneous injection or infusion.

In one embodiment of the present invention the pharmaceutical composition comprises one or more antibodies according to the invention such as monoclonal antibodies together with a pharmaceutical carrier.

Pharmaceutically acceptable carriers include any and all suitable solvents, dispersion media, coatings, antibacterial and antifungal agents, isotonicity agents, antioxidants and absorption-delaying agents, and the like that are physiologically compatible with a compound of the present invention.

Examples of suitable aqueous and non-aqueous carriers which may be employed in the pharmaceutical compositions of the present invention include water, saline, phosphate-buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

The pharmaceutical compositions of the present invention may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The compounds of the present invention may be prepared with carriers that will protect the compound against rapid release, such as a controlled release formulation, including implants, transdermal patches, and micro-encapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, poly-ortho-esters, and polylactic acid alone or with a wax, or other materials well known in the art. Methods for the preparation of such formulations are generally known to those skilled in the art.

In one embodiment, the compounds of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated. Other active or therapeutic compounds may also be incorporated into the compositions.

Pharmaceutical compositions for injection or infusion must typically be sterile and stable under the conditions of manufacture and storage. The composition may be formulated as a solution, micro-emulsion, liposome, or other ordered structure suitable to high drug concentration. The carrier may be an aqueous or a non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum-drying and freeze-drying (lyophilization) that yield a powder of the active ingredient plus any additional desired ingredient from a previously sterile-filtered solution thereof.

Sterile injectable solutions may be prepared by incorporating the active compound i.e. antibody in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum-drying and freeze-drying (lyophilization) that yield a powder of the active ingredient plus any additional desired ingredient from a previously sterile-filtered solution thereof.

The pharmaceutical composition of the present invention may contain one or more monoclonal antibodies or a several monoclonal antibodies such as e.g. polyclonal antibodies of the present invention, a combination of an antibody, a monoclonal antibody or polyclonal antibodies according to the invention with another therapeutic compound, or a combination of compounds of the present invention.

### Methods of the Present Invention

The antibodies of the invention are useful as antimicrobial agents effective against infectious diseases caused by bacteria.

Thus, in one aspect of the present invention comprises a method of treating an individual having an infectious disease by administering to said individual an effective amount of an anti-WTA antibody according to the present invention, e.g an anti-WTA-alpha antibody or an anti-WTA-beta antibody, an anti-CP antibody according to the present invention, e.g. an anti-CP5 antibody or a composition according to the invention.

Examples of such infectious diseases include but are not limited to bacterial lung infections, such as S. aureus pneumonia or tuberculosis infections, bacterial ocular infections, such as trachoma and conjunctivitis, heart, brain or skin infections, infections of the gastrointestinal tract, such as travelers' diarrhea, osteomyelitis, ulcerative colitis, irritable bowel syndrome (IBS), Crohn's disease, and IBD (inflammatory bowel disease) in general, bacterial meningitis, and abscesses in any organ, such as muscle, liver, meninges, or lung. The bacterial infections can also be in other parts of the body like the urinary tract, the bloodstream, a wound or a catheter insertion site.

The antibodies or compositions of the present invention are useful for difficult-to-treat infections that involve biofilms, implants or sanctuary sites (e.g., osteomyelitis and prosthetic joint infections), and high mortality infections such as hospital acquired pneumonia and bacteremia. Vulnerable patient groups that can be treated to prevent Staphylococcal aureus infection include hemodialysis patients, immune-compromised patients, patients in intensive care units, and certain surgical patients.

In another aspect, the invention provides a method of killing, treating, or preventing a microbial infection in an animal, preferably a mammal, and most preferably a human, said method includes administering to the animal, e.g. human, an antibody, composition or pharmaceutical formulation according to the present invention. The invention further features treating or preventing diseases associated with or which opportunistically result from such microbial infections. Such methods of treatment or prevention may include the oral, topical, intravenous, intramuscular, or subcutaneous administration of an antibody or a composition of the invention. For example, prior to surgery or insertion of an IV catheter, in ICU care, in transplant medicine, with or post cancer chemotherapy, or other activities that bear a high risk of infection, the antibody or composition of the present invention may be administered to prevent the onset or spread of infection.

The bacterial infection may be caused by a bacteria with an active and inactive form, and the antibody or composition of the invention may be administered in an amount and for a duration sufficient to treat both the active and the inactive, latent form of the bacterial infection, which duration is longer than is needed to treat the active form of the bacterial infection.

Another aspect of the invention relates to a method of enhancing the effector function of an antibody by introducing a mutation in the Fc region.

Thus in one aspect of the invention relates to a method of enhancing the effector function of an antibody comprising an Fc region and an antigen binding region binding to WTA or CP5, which method comprises introducing a mutation in the Fc region corresponding to position E430, E345 or S440 in human IgG1, EU numbering.

In one embodiment of the invention the method comprises enhancing the effector function of an anti-WTA antibody wherein the Fc region comprises a mutation selected from E430G, E345K, E430S, E430F, E430T, E345Q, E345R, E345Y, S440W or S440Y. In one embodiment of the invention the method comprises enhancing the effector function of an anti-WTA antibody wherein the Fc region comprises a mutation selected from the group consisting of E430G, E345K, E430S, E430F, E430T, E345Q, E345R, E345Y, S440W and S440Y. In a particular embodiment of the invention the method comprises enhancing the effector function of an anti-WTA antibody wherein the Fc region comprises a mutation selected from E430G or E345K. In one embodiment the anti-WTA antibody may be either an anti-WTA-alpha or an anti-WTA-beta antibody.

In one embodiment of the invention the method comprises enhancing the effector function of an anti-CP antibody, wherein the Fc region comprises a mutation selected from E430G, E345K, E430S, E430F, E430T, E345Q, E345R, E345Y, S440W or S440Y. In one embodiment of the invention the method comprises enhancing the effector function of an anti-CP antibody, wherein the Fc region comprises a mutation selected from the group consisting of: E430G, E345K, E430S, E430F, E430T, E345Q, E345R, E345Y, S440W and S440Y. In a particular embodiment of the invention the method comprises enhancing the effector function of an anti-CP antibody, wherein the Fc region comprises a mutation selected from E430G or E345K. In one embodiment the anti-CP antibody is an anti-CP5 antibody

In one embodiment of the invention the effector function is complement activation.

In one embodiment of the invention the effector function is opsonization, such opsonization may be driven by C3 opsonins. In another embodiment of the invention the effector function is antibody induced phagocytosis, such as phagocytosis mediated by immune cells like macrophages and/or neutrophil.

In one embodiment of the invention the effector function is C5a formation and phagocyte activation.

In a further embodiment the effector function is neutrophil-mediated phagocytosis. In one embodiment neutrophil-mediated phagocytosis is determined as disclosed in example 6 or 8. An antibody according the invention is incubated with human serum, fluorescently labeled S. aureus and human neutrophils. Phagocytosis is quantified by flow cytometry.

In one embodiment of the invention the antibody is enhancing phagocytosis in the presence of complement. That is oligomerization of the anti-WTA or anti-CP5 antibodies on the bacteria enhances binding of the complement factor C1q to the Fc region of the antibody creating a C1q:antibody complex, which allows binding of C1q to C1q receptors on phagocytic cells thereby enhancing phagocytosis.

In one embodiment of the invention the antibody enhances phagocytosis of bacteria by immune cells. That is antibodies of the invention may enhance phagocytosis by immune cells such as neutrophils, monocytes, macrophages, kupffer cells, dendritic cells, antigen-presenting cells.

In one embodiment of the invention the antibody is enhancing neutrophil-mediated phagocytosis. Neutrophil-mediated phagocytosis may be determined as in example 6 or 8.

An antibody according the invention is incubated with human serum, fluorescently labeled S. aureus and human neutrophils. Phagocytosis is quantified by flow.

In one embodiment of the invention the antibody enhances complement activation on Gram-positive bacteria. That is in one embodiment the antibody enhances activation of complement protein C4 into C4b. In one embodiment the antibody enhances activation of complement protein C3 into C3b. Activation of C3 on the Gram-positive bacterial cells leads to opsonization of the bacteria by C3-derived opsonins (C3b and C3bi)

In one embodiment of the invention the antibody is enhancing complement-mediated phagocyte activation. That is in one embodiment the antibody enhances formation of the chemoattractant C5a.

In one embodiment of the invention the antibody is enhancing complement-mediated killing.

One aspect of the invention provides for a method of enhancing Fc-Fc contact between antibody molecules on a target cell in vivo comprising;
i) providing an antibody as defined herein and,
ii) bringing the antibody in contact with the antigen on the cell surface of a Gram-positive bacteria in vivo and,
iii) in a concentration that allows Fc-Fc interaction.

Hereby an embodiment of the invention is described that discloses the method of enhancing Fc-Fc contact between antibody molecules of the invention when bound to the target cell in vivo. The term in vivo is to be understood as within the whole living organism such as an animal, including humans.

In one embodiment of the invention the Gram-positive bacteria is Staphylococcus aureus.

In one embodiment of the invention the Gram-positive bacteria is Staphylococcus warneri.

In one embodiment the invention the Gram-positive bacteria is resistant or insensitive to previous treatment with a drug.

In another embodiment of the invention the Gram-positive bacteria is methicillin-resistant S. aureus (MRSA) or methicillin-sensitive S. aureus (MSSA).

### Therapeutic Applications

The antibodies such as monoclonal antibodies or compositions according to any aspect or embodiment of the present invention may be used as a medicament, i.e. for therapeutic applications.

The antibodies may be used for the treatment of humans and other mammals such as cows.

In particular, an anti-WTA antibody or an anti-CP antibody, such as an anti-CP5 antibody, according to the present invention may be used for the treatment of an infectious disease.

In one aspect the anti-WTA antibody of the present invention are used in treatment of Gram-positive bacteria. In one embodiment of the present invention an anti-WTA-alpha antibody is for use in treatment of Gram-positive bacteria. In one embodiment of the present invention an anti-WTA-beta antibody is for use in treatment of Gram-positive bacteria.

In one aspect the anti-CP antibody of the present invention is for use in treatment of an infectious disease. In one aspect the anti-CP antibody of the present invention is for use in treatment of bacteria. In one embodiment of the present invention an anti-CP antibody is for use in treatment of Gram-positive bacteria. In one embodiment of the present invention an anti-CP5 antibody is for use in treatment of an infectious disease. In one embodiment of the present invention an anti-CP5 antibody is for use in treatment of bacteria. In one embodiment of the present invention an anti-CP5 antibody is for use in treatment of Gram-positive bacteria.

In one embodiment of the present invention the anti-WTA antibody or anti-CP antibody such as the anti-CP5 antibody is for use in treatment of a disease caused by Gram-positive bacteria selected from the following group of Staphylococcus, Streptococcus, Bacillus, Clostridium, Corynebacterium, Enterococcus, and Listeria.

In one embodiment of the invention the Staphylococcus is e.g. S. aureus, S. saprophyticus or S. simulans. In one embodiment of the invention the Streptococcus is e.g. S. pneumoniae. In one embodiment of the invention the Clostridium is e.g. C. difficile. In one embodiment the Enterococcus is e.g E. faecalis. In one embodiment of the invention the Listeria is e.g. Listeria monocytogenes.

In one embodiment of the present invention the anti-WTA-beta antibody or anti-WTA-alpha antibody is for use in treatment of S. aureus. In a particular embodiment of the present invention the anti-WTA-beta antibody or anti-WTA-alpha antibody is for use in treatment of MRSA. In one embodiment of the present invention the anti-WTA-beta antibody or anti-WTA-alpha antibody is used in treatment of S. aureus resistant to previous treatment with antibiotics such as trimethoprim-sulfametoxazole (TMP-SMX), clindamycin, doxycycline, minocycline, tetracycline, rifampin, vancomycin or linezolid.

In one embodiment of the present invention the anti-CP antibody, such as an anti-CP5 antibody, is used in treatment of S. aureus. In a particular embodiment of the present invention the anti-CP antibody, such as an anti-CP5 antibody, is used in treatment of MRSA. In one embodiment of the present invention the anti-CP antibody, such as an anti-CP5 antibody, is for use in treatment of S. aureus resistant to previous treatment with antibiotics such as trimethoprim-sulfametoxazole (TMP-SMX), clindamycin, doxycycline, minocycline, tetracycline, rifampin, vancomycin or linezolid.

S. aureus, MSSA and MRSA may cause one or more of the following diseases: Surgical Site Infections (SSI), wound infections, cystic fibrosis, pneumonia, ventilator-associated pneumonia (yap), sepsis, toxic shock syndrome, Intravenous line infections and infections in the presence of prosthetic devices.

In one embodiment of the invention the anti-WTA antibody or anti-CP antibody such as anti-CP5 antibody is use in treatment of a disease selected form the group of: Surgical Site Infections (SSI), wound infections, cystic fibrosis, pneumonia, ventilator-associated pneumonia (yap), sepsis, toxic shock syndrome, Intravenous line infections and infections in the presence of prosthetic devices.

In one embodiment of the invention the anti-WTA antibody or anti-CP antibody such as an anti-CP5 antibody is used in treatment of meningitis, urinary tract infections, pneumoniae.

In one embodiment of the invention the anti-WTA antibody or anti-CP antibody, such as an anti-CP5 antibody, is used in prophylactic treatment of an infection caused by Gram-positive bacteria.

In one embodiment of the invention the anti-WTA antibody or anti-CP antibody, such as an anti-CP5 antibody, is used in adjunctive treatment of an infection caused by Gram-positive.

Hereby are embodiments provided wherein the antibody and/or composition according to the present invention may be administered in combination with an antibiotic.

In one embodiment of the invention the anti-WTA antibody or anti-CP antibody, such as an anti-CP5 antibody, may be used in prevention of patients at risk of developing SSI, VAP or intravascular catheter related infections.

In one embodiment of the invention the anti-WTA antibody or anti-CP antibody, such as an anti-CP5 antibody, may be used in prophylactic treatment of systemic infections such as sepsis or pneumonia.

In one embodiment of the present invention the composition comprises one or more antibodies according to the invention such as monoclonal antibodies for use as a medicament.

### Method of Preparation

It is to be understood that the embodiments described below with reference to an antibody refers to an antibody comprising an Fc region of an immunoglobulin and an antigen-binding region. The antibody may also be a multispecific antibody having a first Fc region of an immunoglobulin and a first antigen-binding region, and a second Fc region of an immunoglobulin and a second antigen-binding region.

The invention also provides isolated nucleic acids and vectors encoding a variant according to any one of the aspects described above, as well as vectors and expression systems encoding the variants. Suitable nucleic acid constructs, vectors and expression systems for antibodies and variants thereof are known in the art, and described in the Examples. In embodiments where the variant comprises not only a heavy chain (or Fc-containing fragment thereof) but also a light chain, the nucleotide sequences encoding the heavy and light chain portions may be present on the same or different nucleic acids or vectors.

The invention also provides a method for producing, in a host cell, an antibody according to any one of the aspects described above, wherein said antibody comprises at least the Fc region of a heavy chain, said method comprising the following steps:

a) providing a nucleotide construct encoding said Fc region of said variant,
b) expressing said nucleotide construct in a host cell, and
c) recovering said antibody variant from a cell culture of said host cell.

In some embodiments, the antibody is a heavy-chain antibody. In most embodiments, however, the antibody will also contain a light chain and thus said host cell further expresses a light-chain-encoding construct, either on the same or a different vector.

Host cells suitable for the recombinant expression of antibodies are well-known in the art, and include CHO, HEK-293, Expi293, PER-C6, NS/0 and Sp2/0 cells. In one embodiment, said host cell is a cell which is capable of Asn-linked glycosylation of proteins, e.g. a eukaryotic cell, such as a mammalian cell, e.g. a human cell. In a further embodiment, said host cell is a non-human cell which is genetically engineered to produce glycoproteins having human-like or human glycosylation. Examples of such cells are genetically-modified Pichia pastoris (Hamilton et al., Science 301 (2003) 1244-1246; Potgieter et al., J. Biotechnology 139 (2009) 318-325) and genetically-modified Lemna minor (Cox et al., Nature Biotechnology 12 (2006) 1591-1597).

In one embodiment, said host cell is a host cell which is not capable of efficiently removing C-terminal lysine K447 residues from antibody heavy chains. For example, Table 2 in Liu et al. (2008) J Pharm Sci 97: 2426 (incorporated herein by reference) lists a number of such antibody production systems, e.g. Sp2/0, NS/0 or transgenic mammary gland (goat), wherein only partial removal of C-terminal lysines is obtained. In one embodiment, the host cell is a host cell with altered glycosylation machinery. Such cells have been described in the art and can be used as host cells in which to express variants of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as EP1176195; WO03/035835; and WO99/54342. Additional methods for generating engineered glycoforms are known in the art, and include but are not limited to those described in Davies et al., 2001, Biotechnol Bioeng 74:288-294; Shields et al, 2002, J Biol Chem 277:26733-26740; Shinkawa et al., 2003, J Biol Chem 278:3466-3473), U.S. Pat. No. 6,602,684, WO00/61739A1; WO01/292246A1; WO02/311140A1; WO 02/30954A1; Potelligent™ μtechnology (Biowa, Inc. Princeton, N.J.); GlycoMAb™ glycosylation engineering technology (GLYCART biotechnology AG, Zurich, Switzerland); US 20030115614; Okazaki et al., 2004, JMB, 336: 1239-49.

The invention also relates to an antibody obtained or obtainable by the method of the invention described above.

In a further aspect, the invention relates to a host cell capable of producing an antibody of the invention. In one embodiment, the host cell has been transformed or transfected with a nucleotide construct of the invention.

The present invention is further illustrated by the following examples which should not be construed as further limiting.

TABLE 1
Sequence table

| SEQ ID NO: | Name | Sequence | Clone |
|---|---|---|---|
| SEQ ID NO: 1 | VH WTA-beta CDR1 | GFSFNSFW | 4497 |
| SEQ ID NO: 2 | VH WTA-beta CDR2 | TNNEGTTT | |
| SEQ ID NO: 3 | VH WTA-beta CDR3 | ARGDGGLDD | |
| SEQ ID NO: 4 | VH WTA-beta | EVQLVESGGGLVQPGGSLRLSCSA | |
| | | SGFSFNSFWMHWVRQVPGKGL | |
| | | VWISFTNNEGTTTAYADSVRGRFI | |
| | | ISRDNAKNTLYLEMNNLRGEDTA | |
| | | VYYCARGDGGLDDWGQGTLVTV | |
| | | SS | |
| SEQ ID NO: 5 | VL WTA-beta CDR1 | QSIFRTSRNKNL | |
| | VL WTA-beta CDR2 | WAS | |
| SEQ ID NO: 6 | VL WTA-beta CDR3 | QQYFSPPYT | |
| SEQ ID NO: 7 | VL WTA-beta | DIQLTQSPDSLAVSLGERATINCKS | |
| | | SQSIFRTSRNKNLLNWYQQRPGQ | |
| | | PPRLLIHWASTRKSGVPDRFSGSG | |
| | | FGTDFTLTITSLQAEDVAIYYCQQY | |
| | | FSPPYTFGQGTKLEIK | |
| SEQ ID NO: 8 | VH CP5 CDR1 | GFSLTNYG | 137G18A- |
| | | | N107D |
| SEQ ID NO: 9 | VH CP5 CDR2 | IWSGGNT | |
| SEQ ID NO: 10 | VH CP5 CDR3 | ARDRYDVRAFDY | |
| SEQ ID NO: 11 | VH CP5 | QVQLKQSGPGLVQPSQSLSITCTV | |
| | | SGFSLTNYGVHWVRQSPGKGLE | |
| | | WLGVIWSGGNTDYNAAFISRLSIS | |
| | | KDNSKSQVFFKMNSLQADDTAIY | |
| | | YCARDRYDVRAFDYWGQGTTLT | |
| | | VSS | |
| SEQ ID NO: 12 | VL CP5 CDR1 | QNVRTA | |
| | VL CP5 CDR2 | LAS | |
| SEQ ID NO: 13 | VL CP5 CDR3 | LQHWNYLYT | |
| SEQ ID NO: 14 | VL CP5 | DIVMTQSQKFMSTSVGDRVSITC | |
| | | KASQNVRTAVAWYQQKPGQSPK | |
| | | ALIYLASNRHTGVPDRFTGSGSGT | |
| | | DFTLTISNVQSEDLADYFCLQHW | |
| | | NYLYTFGGGTKLEIKK | |
| SEQ ID NO: 15 | Fc IgG1f-E430G | ASTKGPSVFPLAPSSKSTSGGTAAL | |
| | | GCLVKDYFPEPVTVSWNSGALTS | |
| | | GVHTFPAVLQSSGLYSLSSVVTVP | |
| | | SSSLGTQTYICNVNHKPSNTKVDK | |
| | | RVEPKSCDKTHTCPPCPAPELLGG | |
| | | PSVFLFPPKPKDTLMISRTPEVTCV | |
| | | WDVSHEDPEVKFNWYVDGVEV | |
| | | HNAKTKPREEQYNSTYRVVSVLTV | |
| | | LHQDWLNGKEYKCKVSNKALPAP | |
| | | IEKTISKAKGQPREPQVYTLPPSRE | |
| | | EMTKNQVSLTCLVKGFYPSDIAVE | |
| | | WESNGQPENNYKTTPPVLDSDGS | |
| | | FFLYSKLTVDKSRWQQGNVFSCS | |
| | | VMHGALHNHYTQKSLSLSPGK | |
| SEQ ID NO: 16 | Fc IgG1f-E345K | ASTKGPSVFPLAPSSKSTSGGTAAL | |
| | | GCLVKDYFPEPVTVSWNSGALTS | |
| | | GVHTFPAVLQSSGLYSLSSVVTVP | |
| | | SSSLGTQTYICNVNHKPSNTKVDK | |
| | | RVEPKSCDKTHTCPPCPAPELLGG | |
| | | PSVFLFPPKPKDTLMISRTPEVTCV | |
| | | WDVSHEDPEVKFNWYVDGVEV | |
| | | HNAKTKPREEQYNSTYRVVSVLTV | |
| | | LHQDWLNGKEYKCKVSNKALPAP | |
| | | IEKTISKAKGQPRKPQVYTLPPSRE | |
| | | EMTKNQVSLTCLVKGFYPSDIAVE | |
| | | WESNGQPENNYKTTPPVLDSDGS | |
| | | FFLYSKLTVDKSRWQQGNVFSCS | |
| | | VMHEALHNHYTQKSLSLSPGK | |
| SEQ ID NO: 17 | Fc IgG1f-E430S | ASTKGPSVFPLAPSSKSTSGGTAAL | |
| | | GCLVKDYFPEPVTVSWNSGALTS | |
| | | GVHTFPAVLQSSGLYSLSSVVTVP | |
| | | SSSLGTQTYICNVNHKPSNTKVDK | |
| | | RVEPKSCDKTHTCPPCPAPELLGG | |
| | | PSVFLFPPKPKDTLMISRTPEVTCV | |
| | | WDVSHEDPEVKFNWYVDGVEV | |
| | | HNAKTKPREEQYNSTYRVVSVLTV | |
| | | LHQDWLNGKEYKCKVSNKALPAP | |
| | | IEKTISKAKGQPREPQVYTLPPSRE | |
| | | EMTKNQVSLTCLVKGFYPSDIAVE | |
| | | WESNGQPENNYKTTPPVLDSDGS | |
| | | FFLYSKLTVDKSRWQQGNVFSCS | |
| | | VMHSALHNHYTQKSLSLSPGK | |
| SEQ ID NO: 18 | Fc IgG1f-E430F | ASTKGPSVFPLAPSSKSTSGGTAAL | |
| | | GCLVKDYFPEPVTVSWNSGALTS | |
| | | GVHTFPAVLQSSGLYSLSSVVTVP | |
| | | SSSLGTQTYICNVNHKPSNTKVDK | |
| | | RVEPKSCDKTHTCPPCPAPELLGG | |
| | | PSVFLFPPKPKDTLMISRTPEVTCV | |
| | | WDVSHEDPEVKFNWYVDGVEV | |
| | | HNAKTKPREEQYNSTYRVVSVLTV | |
| | | LHQDWLNGKEYKCKVSNKALPAP | |
| | | IEKTISKAKGQPREPQVYTLPPSRE | |
| | | EMTKNQVSLTCLVKGFYPSDIAVE | |
| | | WESNGQPENNYKTTPPVLDSDGS | |
| | | FFLYSKLTVDKSRWQQGNVFSCS | |
| | | VMHFALHNHYTQKSLSLSPGK | |
| SEQ ID NO: 19 | Fc IgG1f-E430T | ASTKGPSVFPLAPSSKSTSGGTAAL | |
| | | GCLVKDYFPEPVTVSWNSGALTS | |
| | | GVHTFPAVLQSSGLYSLSSVVTVP | |
| | | SSSLGTQTYICNVNHKPSNTKVDK | |
| | | RVEPKSCDKTHTCPPCPAPELLGG | |
| | | PSVFLFPPKPKDTLMISRTPEVTCV | |
| | | WDVSHEDPEVKFNWYVDGVEV | |
| | | HNAKTKPREEQYNSTYRVVSVLTV | |
| | | LHQDWLNGKEYKCKVSNKALPAP | |
| | | IEKTISKAKGQPREPQVYTLPPSRE | |
| | | EMTKNQVSLTCLVKGFYPSDIAVE | |
| | | WESNGQPENNYKTTPPVLDSDGS | |
| | | FFLYSKLTVDKSRWQQGNVFSCS | |
| | | VMHTALHNHYTQKSLSLSPGK | |
| SEQ ID NO: 20 | Fc IgG1f-E345Q | ASTKGPSVFPLAPSSKSTSGGTAAL | |
| | | GCLVKDYFPEPVTVSWNSGALTS | |
| | | GVHTFPAVLQSSGLYSLSSVVTVP | |
| | | SSSLGTQTYICNVNHKPSNTKVDK | |
| | | RVEPKSCDKTHTCPPCPAPELLGG | |
| | | PSVFLFPPKPKDTLMISRTPEVTCV | |
| | | WDVSHEDPEVKFNWYVDGVEV | |
| | | HNAKTKPREEQYNSTYRVVSVLTV | |
| | | LHQDWLNGKEYKCKVSNKALPAP | |
| | | IEKTISKAKGQPRQPQVYTLPPSRE | |
| | | EMTKNQVSLTCLVKGFYPSDIAVE | |
| | | WESNGQPENNYKTTPPVLDSDGS | |
| | | FFLYSKLTVDKSRWQQGNVFSCS | |
| | | VMHEALHNHYTQKSLSLSPGK | |
| SEQ ID NO: 21 | Fc IgG1f-E345R | ASTKGPSVFPLAPSSKSTSGGTAAL | |
| | | GCLVKDYFPEPVTVSWNSGALTS | |
| | | GVHTFPAVLQSSGLYSLSSVVTVP | |
| | | SSSLGTQTYICNVNHKPSNTKVDK | |
| | | RVEPKSCDKTHTCPPCPAPELLGG | |
| | | PSVFLFPPKPKDTLMISRTPEVTCV | |
| | | WDVSHEDPEVKFNWYVDGVEV | |
| | | HNAKTKPREEQYNSTYRVVSVLTV | |
| | | LHQDWLNGKEYKCKVSNKALPAP | |
| | | IEKTISKAKGQPRRPQVYTLPPSRE | |
| | | EMTKNQVSLTCLVKGFYPSDIAVE | |
| | | WESNGQPENNYKTTPPVLDSDGS | |
| | | FFLYSKLTVDKSRWQQGNVFSCS | |
| | | VMHEALHNHYTQKSLSLSPGK | |
| SEQ ID NO: 22 | Fc IgG1f-E345Y | ASTKGPSVFPLAPSSKSTSGGTAAL | |
| | | GCLVKDYFPEPVTVSWNSGALTS | |
| | | GVHTFPAVLQSSGLYSLSSVVTVP | |
| | | SSSLGTQTYICNVNHKPSNTKVDK | |
| | | RVEPKSCDKTHTCPPCPAPELLGG | |
| | | PSVFLFPPKPKDTLMISRTPEVTCV | |
| | | WDVSHEDPEVKFNWYVDGVEV | |
| | | HNAKTKPREEQYNSTYRVVSVLTV | |
| | | LHQDWLNGKEYKCKVSNKALPAP | |
| | | IEKTISKAKGQPRYPQVYTLPPSRE | |
| | | EMTKNQVSLTCLVKGFYPSDIAVE | |
| | | WESNGQPENNYKTTPPVLDSDGS | |
| | | FFLYSKLTVDKSRWQQGNVFSCS | |
| | | VMHEALHNHYTQKSLSLSPGK | |
| SEQ ID NO: 23 | Fc IgG1f-S440W | ASTKGPSVFPLAPSSKSTSGGTAAL | |
| | | GCLVKDYFPEPVTVSWNSGALTS | |
| | | GVHTFPAVLQSSGLYSLSSVVTVP | |
| | | SSSLGTQTYICNVNHKPSNTKVDK | |
| | | RVEPKSCDKTHTCPPCPAPELLGG | |
| | | PSVFLFPPKPKDTLMISRTPEVTCV | |
| | | WDVSHEDPEVKFNWYVDGVEV | |
| | | HNAKTKPREEQYNSTYRVVSVLTV | |
| | | LHQDWLNGKEYKCKVSNKALPAP | |
| | | IEKTISKAKGQPREPQVYTLPPSRE | |
| | | EMTKNQVSLTCLVKGFYPSDIAVE | |
| | | WESNGQPENNYKTTPPVLDSDGS | |
| | | FFLYSKLTVDKSRWQQGNVFSCS | |
| | | VMHEALHNHYTQKWLSLSPGK | |
| SEQ ID NO: 24 | Fc IgG1f-S440Y | ASTKGPSVFPLAPSSKSTSGGTAAL | |
| | | GCLVKDYFPEPVTVSWNSGALTS | |
| | | GVHTFPAVLQSSGLYSLSSVVTVP | |
| | | SSSLGTQTYICNVNHKPSNTKVDK | |
| | | RVEPKSCDKTHTCPPCPAPELLGG | |
| | | PSVFLFPPKPKDTLMISRTPEVTCV | |
| | | VVDVSHEDPEVKFNWYVDGVEV | |
| | | HNAKTKPREEQYNSTYRVVSVLTV | |
| | | LHQDWLNGKEYKCKVSNKALPAP | |
| | | IEKTISKAKGQPREPQVYTLPPSRE | |
| | | EMTKNQVSLTCLVKGFYPSDIAVE | |
| | | WESNGQPENNYKTTPPVLDSDGS | |
| | | FFLYSKLTVDKSRWQQGNVFSCS | |
| | | VMHEALHNHYTQKYLSLSPGK | |
| SEQ ID NO: 25 | Fc IgG2-E430G | ASTKGPSVFPLAPCSRSTSESTAAL | |
| | | GCLVKDYFPEPVTVSWNSGALTS | |
| | | GVHTFPAVLQSSGLYSLSSVVTVP | |
| | | SSNFGTQTYTCNVDHKPSNTKVD | |
| | | KTVERKCCVECPPCPAPPVAGPSV | |
| | | FLFPPKPKDTLMISRTPEVTCVVV | |
| | | DVSHEDPEVQFNWYVDGVEVHN | |
| | | AKTKPREEQFNSTFRVVSVLTVVH | |
| | | QDWLNGKEYKCKVSNKGLPAPIE | |
| | | KTISKTKGQPREPQVYTLPPSREE | |
| | | MTKNQVSLTCLVKGFYPSDIAVE | |
| | | WESNGQPENNYKTTPPMLDSDG | |
| | | SFFLYSKLTVDKSRWQQGNVFSCS | |
| | | VMHGALHNHYTQKSLSLSPGK | |
| SEQ ID NO: 26 | Fc IgG2-E345K | ASTKGPSVFPLAPCSRSTSESTAAL | |
| | | GCLVKDYFPEPVTVSWNSGALTS | |
| | | GVHTFPAVLQSSGLYSLSSVVTVP | |
| | | SSNFGTQTYTCNVDHKPSNTKVD | |
| | | KTVERKCCVECPPCPAPPVAGPSV | |
| | | FLFPPKPKDTLMISRTPEVTCVVV | |
| | | DVSHEDPEVQFNWYVDGVEVHN | |
| | | AKTKPREEQFNSTFRVVSVLTVVH | |
| | | QDWLNGKEYKCKVSNKGLPAPIE | |
| | | KTISKTKGQPRKPQVYTLPPSREE | |
| | | MTKNQVSLTCLVKGFYPSDIAVE | |
| | | WESNGQPENNYKTTPPMLDSDG | |
| | | SFFLYSKLTVDKSRWQQGNVFSCS | |
| | | VMHEALHNHYTQKSLSLSPGK | |
| SEQ ID NO: 27 | Fc IgG2-E430S | ASTKGPSVFPLAPCSRSTSESTAAL | |
| | | GCLVKDYFPEPVTVSWNSGALTS | |
| | | GVHTFPAVLQSSGLYSLSSVVTVP | |
| | | SSNFGTQTYTCNVDHKPSNTKVD | |
| | | KTVERKCCVECPPCPAPPVAGPSV | |
| | | FLFPPKPKDTLMISRTPEVTCVVV | |
| | | DVSHEDPEVQFNWYVDGVEVHN | |
| | | AKTKPREEQFNSTFRVVSVLTVVH | |
| | | QDWLNGKEYKCKVSNKGLPAPIE | |
| | | KTISKTKGQPREPQVYTLPPSREE | |
| | | MTKNQVSLTCLVKGFYPSDIAVE | |
| | | WESNGQPENNYKTTPPMLDSDG | |
| | | SFFLYSKLTVDKSRWQQGNVFSCS | |
| | | VMHSALHNHYTQKSLSLSPGK | |
| SEQ ID NO: 28 | Fc IgG2-E430F | ASTKGPSVFPLAPCSRSTSESTAAL | |
| | | GCLVKDYFPEPVTVSWNSGALTS | |
| | | GVHTFPAVLQSSGLYSLSSVVTVP | |
| | | SSNFGTQTYTCNVDHKPSNTKVD | |
| | | KTVERKCCVECPPCPAPPVAGPSV | |
| | | FLFPPKPKDTLMISRTPEVTCVVV | |
| | | DVSHEDPEVQFNWYVDGVEVHN | |
| | | AKTKPREEQFNSTFRVVSVLTVVH | |
| | | QDWLNGKEYKCKVSNKGLPAPIE | |
| | | KTISKTKGQPREPQVYTLPPSREE | |
| | | MTKNQVSLTCLVKGFYPSDIAVE | |
| | | WESNGQPENNYKTTPPMLDSDG | |
| | | SFFLYSKLTVDKSRWQQGNVFSCS | |
| | | VMHFALHNHYTQKSLSLSPGK | |
| SEQ ID NO: 29 | Fc IgG2-E430T | ASTKGPSVFPLAPCSRSTSESTAAL | |
| | | GCLVKDYFPEPVTVSWNSGALTS | |
| | | GVHTFPAVLQSSGLYSLSSVVTVP | |
| | | SSNFGTQTYTCNVDHKPSNTKVD | |
| | | KTVERKCCVECPPCPAPPVAGPSV | |
| | | FLFPPKPKDTLMISRTPEVTCVVV | |
| | | DVSHEDPEVQFNWYVDGVEVHN | |
| | | AKTKPREEQFNSTFRVVSVLTVVH | |
| | | QDWLNGKEYKCKVSNKGLPAPIE | |
| | | KTISKTKGQPREPQVYTLPPSREE | |
| | | MTKNQVSLTCLVKGFYPSDIAVE | |
| | | WESNGQPENNYKTTPPMLDSDG | |
| | | SFFLYSKLTVDKSRWQQGNVFSCS | |
| | | VMHTALHNHYTQKSLSLSPGK | |
| SEQ ID NO: 30 | Fc IgG2-E345Q | ASTKGPSVFPLAPCSRSTSESTAAL | |
| | | GCLVKDYFPEPVTVSWNSGALTS | |
| | | GVHTFPAVLQSSGLYSLSSVVTVP | |
| | | SSNFGTQTYTCNVDHKPSNTKVD | |
| | | KTVERKCCVECPPCPAPPVAGPSV | |
| | | FLFPPKPKDTLMISRTPEVTCVVV | |
| | | DVSHEDPEVQFNWYVDGVEVHN | |
| | | AKTKPREEQFNSTFRVVSVLTVVH | |
| | | QDWLNGKEYKCKVSNKGLPAPIE | |
| | | KTISKTKGQPRQPQVYTLPPSREE | |
| | | MTKNQVSLTCLVKGFYPSDIAVE | |
| | | WESNGQPENNYKTTPPMLDSDG | |
| | | SFFLYSKLTVDKSRWQQGNVFSCS | |
| | | VMHEALHNHYTQKSLSLSPGK | |
| SEQ ID NO: 31 | Fc IgG2-E345R | ASTKGPSVFPLAPCSRSTSESTAAL | |
| | | GCLVKDYFPEPVTVSWNSGALTS | |
| | | GVHTFPAVLQSSGLYSLSSVVTVP | |
| | | SSNFGTQTYTCNVDHKPSNTKVD | |
| | | KTVERKCCVECPPCPAPPVAGPSV | |
| | | FLFPPKPKDTLMISRTPEVTCVVV | |
| | | DVSHEDPEVQFNWYVDGVEVHN | |
| | | AKTKPREEQFNSTFRVVSVLTVVH | |
| | | QDWLNGKEYKCKVSNKGLPAPIE | |
| | | KTISKTKGQPRRPQVYTLPPSREE | |
| | | MTKNQVSLTCLVKGFYPSDIAVE | |
| | | WESNGQPENNYKTTPPMLDSDG | |
| | | SFFLYSKLTVDKSRWQQGNVFSCS | |
| | | VMHEALHNHYTQKSLSLSPGK | |
| SEQ ID NO: 32 | Fc IgG2-E345Y | ASTKGPSVFPLAPCSRSTSESTAAL | |
| | | GCLVKDYFPEPVTVSWNSGALTS | |
| | | GVHTFPAVLQSSGLYSLSSVVTVP | |
| | | SSNFGTQTYTCNVDHKPSNTKVD | |
| | | KTVERKCCVECPPCPAPPVAGPSV | |
| | | FLFPPKPKDTLMISRTPEVTCVVV | |
| | | DVSHEDPEVQFNWYVDGVEVHN | |
| | | AKTKPREEQFNSTFRVVSVLTVVH | |
| | | QDWLNGKEYKCKVSNKGLPAPIE | |
| | | KTISKTKGQPRYPQVYTLPPSREE | |
| | | MTKNQVSLTCLVKGFYPSDIAVE | |
| | | WESNGQPENNYKTTPPMLDSDG | |
| | | SFFLYSKLTVDKSRWQQGNVFSCS | |
| | | VMHEALHNHYTQKSLSLSPGK | |
| SEQ ID NO: 33 | Fc IgG2-S440W | ASTKGPSVFPLAPCSRSTSESTAAL | |
| | | GCLVKDYFPEPVTVSWNSGALTS | |
| | | GVHTFPAVLQSSGLYSLSSVVTVP | |
| | | SSNFGTQTYTCNVDHKPSNTKVD | |
| | | KTVERKCCVECPPCPAPPVAGPSV | |
| | | FLFPPKPKDTLMISRTPEVTCVVV | |
| | | DVSHEDPEVQFNWYVDGVEVHN | |
| | | AKTKPREEQFNSTFRVVSVLTVVH | |
| | | QDWLNGKEYKCKVSNKGLPAPIE | |
| | | KTISKTKGQPREPQVYTLPPSREE | |
| | | MTKNQVSLTCLVKGFYPSDIAVE | |
| | | WESNGQPENNYKTTPPMLDSDG | |
| | | SFFLYSKLTVDKSRWQQGNVFSCS | |
| | | VMHEALHNHYTQKWLSLSPGK | |
| SEQ ID NO: 34 | Fc IgG2-S440Y | ASTKGPSVFPLAPCSRSTSESTAAL | |
| | | GCLVKDYFPEPVTVSWNSGALTS | |
| | | GVHTFPAVLQSSGLYSLSSVVTVP | |
| | | SSNFGTQTYTCNVDHKPSNTKVD | |
| | | KTVERKCCVECPPCPAPPVAGPSV | |
| | | FLFPPKPKDTLMISRTPEVTCVVV | |
| | | DVSHEDPEVQFNWYVDGVEVHN | |
| | | AKTKPREEQFNSTFRVVSVLTVVH | |
| | | QDWLNGKEYKCKVSNKGLPAPIE | |
| | | KTISKTKGQPREPQVYTLPPSREE | |
| | | MTKNQVSLTCLVKGFYPSDIAVE | |
| | | WESNGQPENNYKTTPPMLDSDG | |
| | | SFFLYSKLTVDKSRWQQGNVFSCS | |
| | | VMHEALHNHYTQKYLSLSPGK | |
| SEQ ID NO: 35 | VH WTA-beta CDR1 | GYTFTSYD | 6297 |
| SEQ ID NO: 36 | VH WTA-beta CDR2 | MNPNSGNT | |
| SEQ ID NO: 37 | VH WTA-beta CDR3 | ATERWSKDTGHYYYYGMDV | |
| SEQ ID NO: 38 | VH WTA-beta | QVQLQQSGAEVKKPGASVKV | |
| | | SCRASGYTFTSYDINWVRQA | |
| | | PGQGLEWMGWMNPNSGNTNY | |
| | | AQRFQGRLTMTKNTSINTAY | |
| | | MELSSLRSEDTAVYYCATER | |
| | | WSKDTGHYYYYGMDVWGQGT | |
| | | TVTVSSASTKGPSVFPLAPS | |
| | | SKSTSGGTAALGCLVKDYFP | |
| | | EPVTVSWNSGALTSGVHTFP | |
| | | AVLQSSGLYSLSSVVTVPSS | |
| | | SLGTQTYICNVNHKPSNTKV | |
| | | DKKVEPKSCDKTHTCPPCPA | |
| | | PELLGGPSVFLFPPKPKDTL | |
| | | MISRTPEVTCVVVDVSHEDP | |
| | | EVKFNWYVDGVEVHNAKTKP | |
| | | REEQYNSTYRVVSVLTVLHQ | |
| | | DWLNGKEYKCKVSNKALPAP | |
| | | IEKTISKAKGQPREPQVYTL | |
| | | PPSREEMTKNQVSLTCLVKG | |
| | | FYPSDIAVEWESNGQPENNY | |
| | | KTTPPVLDSDGSFFLYSKLT | |
| | | VDKSRWQQGNVFSCSVMHEA | |
| | | LHNHYTQKSLSLSPG | |
| SEQ ID NO: 39 | VL WTA-beta CDR1 | QSVSSSY | |
| | VL WTA-beta CDR2 | DAS | |
| SEQ ID NO: 40 | VL WTA-beta CDR3 | QKYGSTPRP | |
| SEQ ID NO: 41 | VL WTA-beta | ETTLTQSPGTLSLSPGERAT | |
| | | LSCRASQSVSSSYLAWYQQK | |
| | | PGQAPKVLIYDASSRATGIP | |
| | | DRFSGSGSGTDFTLTISRLE | |
| | | PEDFAVYYCQKYGSTPRPFGQGT | |
| | | KVEIKRTVAAPSVFIFP | |
| | | PSDEQLKSGTASVVCLLNNF | |
| | | YPREAKVQWKVDNALQSGNS | |
| | | QESVTEQDSKDSTYSLSSTL | |
| | | TLSKADYEKHKVYACEVTHQ | |
| | | GLSSPVTKSFNRGEC | |

The CDR regions have been annotated according to the IMGT definitions.

### EXAMPLES

### Example 1: Antibodies and Peptides

### Expression Constructs for Antibodies

For monoclonal antibody (mAb) expression variable heavy (VH) chain and variable light (VL) chain sequences were cloned in pcDNA3.3 expression vectors containing human IgG1 or IgG2 heavy chain (HC) and light chain (LC) constant regions as indicated in the examples. Desired mutations were introduced either by gene synthesis or site directed mutagenesis. Anti-MRSA Antibodies mentioned in this application have VH and VL sequences derived from previously described antibodies: human mAbs anti-wall teichoid acid GlcNAc beta 4497 (anti-WTA 4497; based on WO2014/193722) and anti-WTA IgG1-6297 (based on WO2014/193722), humanized mAb anti-CIfA tefibazumab (based on WO2002/072600) and mouse mAb anti-capsular polysaccharide type 5 (anti-CP5; based on WO2014/027698). In some of the examples the human antibody IgG1-b12 against HIV gp120 was used as a non-binding isotype control (Barbas et al., J Mol Biol. 1993 Apr. 5; 230(3):812-23).

### Transient Expression

Antibodies were expressed as IgG1,κ or IgG2,κ. Plasmid DNA mixtures encoding both heavy and light chains of antibodies were transiently transfected in Expi293T cells (Life technologies, USA) using 293fectin (Life technologies) essentially as described by Vink et al. (Vink et al., Methods, 65 (1), 5-10 2014).

### Purification and Analysis of Proteins

Antibodies were purified by immobilized protein G chromatography and batches were analyzed by a number of bioanalytical assays including SDS-PAGE and size exclusion chromatography.

### Peptides

The Fc-III peptide DCAWHLGELVWCT (deLano et al., 2000 Science), scrambled versions of the Fc-III sequence (Fc-III Scrambled 1: ACWTLEWGVLDCH; Fc-III Scrambled 2: WCDLEGVTWHACL) and a control peptide (GWTVFQKRLDGSV) were synthesized by Pepscan (Lelystad, The Netherlands). Peptides were dissolved in MiliQ water at 1 or 2 mg/mLI and stored in small aliquots at −80° C.

### Example 2: Inhibition of Fc-Fc Interactions Results in Decreased Induction of Complement Deposition on the Bacterial Surface by Naturally Occurring Antibodies Against S. aureus

The effect of the competing Fc-binding peptide on complement activation by antibodies against S. aureus was tested by measuring C4b and C3b deposition on S. aureus bacteria of the non-Protein A-bearing Wood 46 strain after opsonization with naturally occurring antibodies present in normal human serum (NHS) in the presence or absence of the peptide. C4b is the first complement component covalently deposited on the bacterial surface by C1.

As a source of complement and naturally occurring human antibodies against S. aureus, normal human serum (NHS) from 20 healthy donors was pooled. Venous blood from healthy volunteers was collected at the Mini Donor Dienst (MDD) of the UMC Utrecht (METC-protocol 07-125/C approved Mar. 1, 2010) in 9 mL BD Vacutainer blood tubes containing a clot activator (BD; Cat #367896). Clotting was allowed for 15 minutes (min) at room temperature (RT) without agitation and serum was collected by centrifugation at 2080g for 20 min at 4° C. Sera of 20 healthy volunteers was pooled and aliquots (100 to 1000 μL in Eppendorf tubes) stored at −80° C. Heat-inactivated (HI) serum was prepared with thawed serum by incubation at 56° C. for 30 min.

Wood 46 bacteria (ATCC #10832) were grown overnight on blood agar plates at 37° C. Bacteria were collected with an inoculation loop into PBS, photometrical (OD 660 nm) adjusted to a concentration of 5×10⁸/mL, washed and resuspended in HEPES buffer (20 mM HEPES, 140 mM NaCl) containing 5 mM CaCl₂ and 2.5 mM MgCl₂ (HEPES++), supplemented with 0.5% bovine serum albumin (BSA; Serva; Cat #11930.03) at pH 7.3. For opsonization, 50 μL washed bacteria (5×10⁸/mL) were incubated with 50 μL peptide pre-incubated NHS in round-bottom 96-well microplates (Greiner; Cat #650101) for 20 min at 37° C. while shaking at 600 rpm. In one experiment, a concentration series of NHS (range 0.075 to 5% in 2-fold dilutions) was pre-incubated with 20 μg/mL Fc-III peptide or control peptide, while in the other a fixed concentration of 1% NHS was pre-incubated with a concentration series (range 40 to 0.0375 μg/mL in 2-fold dilutions) of Fc-III peptide, Fc-III scrambled 1, or Fc-III scrambled 2 peptide. All peptide pre-incubations were performed for 10 min at RT without shaking. Bacteria were washed twice with PBS with 1% BSA (PBSA) and incubated with 50 μL of 1 μg/mL mouse-anti-human C4d (Quidel, Cat #A213) or mouse-anti-human C3d antibody (Quidel, Cat #A207) for 45 min at 4° C. while shaking at 600 rpm. Bacteria were washed twice with PBSA and incubated with 50 μL of 1 μg/mL FITC-conjugated Goat F(ab′)2 anti-mouse Immunoglobulins antibody (Dako, Cat #F0479) for 45 min at 4° C. while shaking at 600 rpm. Samples were washed, fixed with 150 μL 1% paraformaldehyde in PBS (PFA; 10% methanol-free Formaldehyde, Polysciences Cat #04018) and analyzed on a FACSVerse flow cytometer (BD) equipped with a universal loader for 96-well microplates. Samples were acquired with low event thresholds on both forward scatter (FSC) and side scatter (SSC) in log-mode. Pseudocolor FSC versus SSC density plots were used to gate single bacteria as the highest relative population density and to determine their mean fluorescence intensity (MFI). The flow cytometry data were analyzed by FlowJo software version 10.0.7.

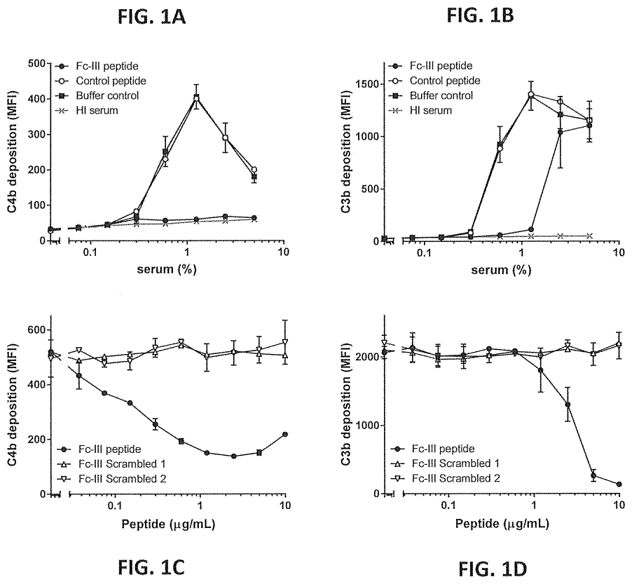

FIG. 1 shows that in the absence of peptide (buffer control), incubation of S. aureus Wood 46 with serum resulted in dose-dependent C4b (FIG. 1A) and C3b (FIG. 1B) deposition on the bacterial surface. In the presence of the Fc-III peptide, complement deposition was strongly inhibited, with C4d levels as low as basic levels observed with heat-inactivated (HI) serum, which contains no active complement, at all tested serum concentrations and C3b levels as low as basic levels observed with HI serum in the serum concentration range up to 1%. In contrast, the negative control peptide had no effect on C4b and C3b deposition. Using a fixed concentration of 1% serum, the Fc-III peptide resulted in dose-dependent inhibition of C4b (FIG. 1C) and C3b (FIG. 1D) deposition, while the non-specific scrambled peptides showed no inhibitory effects.

In conclusion, the observed inhibition of C4b and C3b deposition by the competing Fc-III peptide DCAWHLGELVWCT suggests that naturally occurring antibodies in human sera establish Fc-Fc interactions involved in IgG hexamerization to induce classical complement pathway activation on non-Protein A-bearing Wood 46 S. aureus bacteria.

### Example 3: Inhibition of Fc-Fc Interactions Results in Decreased Induction of Phagocytic Uptake of Bacteria by Naturally Occurring Antibodies Against S. aureus

In humans, host clearance of S. aureus critically depends on proper engulfment and intracellular killing by phagocytic cells that are most potently recruited through binding of their complement receptors (CD35, CD11b/CD18) to C3b/iC3b molecules deposited on the bacterial surface after complement activation. To test if inhibition of C4b and C3b deposition by the Fc-III peptide as described in Example 2 affects phagocytic uptake of the bacteria, fluorescently labeled Wood 46 bacteria were incubated with human neutrophils after opsonization with naturally occurring antibodies present in NHS in the presence or absence of the peptide.

Wood 46 bacteria were fluorescein isothiocyanate (FITC)-labeled. Therefore, bacteria were grown overnight on blood agar plates at 37° C. and collected into PBS. Bacteria were washed by centrifugation for 15 min at 3000×g and suspension in 10 mL PBS. FITC Isomer I (Sigma, Cat #F4274; dissolved at 10 mg/mL in DMSO) was added at 0.5 mg/mL and incubated for 30 min on ice. Bacteria were washed twice with 10 mL PBS and resuspended in 1×RPMI medium 1640 with L-Glutamine and 25 mM HEPES (Gibco Life Technologies, Cat #52400) supplemented with 0.05% human serum albumin (HSA; Albuman 200 g/L for iv use from Sanquin). Bacterial concentration was determined photospectrometrically at 660 nm and aliquots of 1×10⁹ c/mL were stored at −20° C. in 1.5 mL Eppendorf tubes. 20 μL of a concentration series (0.01 to 10% in 3-fold dilutions) of pooled NHS containing naturally occurring antibodies against S. aureus was pre-incubated for 10 min at RT with 10 μL of 0, 5, 10 or 20 μg/mL Fc-III peptide or 20 μg/mL control peptide in RPMI-0.05% HSA. 20 μL of 3.75×10⁷/mL FITC-labeled Wood 46 bacteria was added and incubated for 20 min at 37° C. while shaking (600 rpm). For the isolation of human neutrophils, blood from a healthy donor was collected in Vacuette NH Sodium Heparin vacutainers (Greiner Bio-one, Cat #455051) and neutrohils were isolated by Ficoll-Histopaque gradient (Ficoll-Paque PLUS, GE Healthcare Lifesciences, Cat #17-1440-03; Histopaque 1119, Sigma, Cat #11191; Bestebroer et al., 2007 Blood 109: 2936-2943) and suspended in RPMI-HSA. 10 μL of 7.5×10⁶/mL neutrophils were added to the opsonized bacteria and phagocytosis was allowed for 15 min at 37° C. while shaking at 600 rpm. 90 μL cold paraformaldehyde (1.7% in RPMI-HSA) was added to stop the reaction and samples were analyzed by flow cytometry (FACSVerse, BD) measurement of the fluorescence of the neutrophils. The neutrophil population was gated in a pseudocolor density plot of FSC versus SSC to exclude cell debris and the population analyzed for their MFI representing associated fluorescent bacteria.

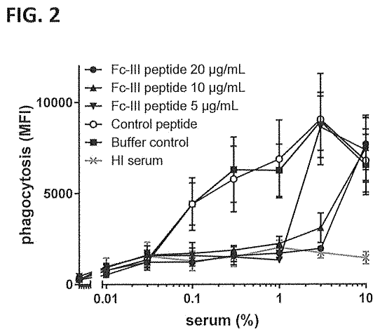

FIG. 2 shows that in NHS, FITC-labeled bacteria were taken up by neutrophils in a dose-dependent manner (buffer control). In contrast, no phagocytosis was observed in HI serum, suggesting that complement activation is required for the neutrophil-mediated phagocytosis. In the presence of the competing Fc-III peptide, phagocytosis was potently blocked, whereas the control peptide had no effect.

In conclusion, inhibition of neutrophil-mediated phagocytosis by the competing Fc-III peptide DCAWHLGELVWCT suggests that naturally occurring antibodies against S. aureus establish Fc-Fc interactions involved in IgG hexamerization to induce complement-mediated phagocytosis of non-Protein A-bearing S. aureus Wood 46 bacteria by neutrophils.

### Example 4: Inhibition of Fc-Fc Interactions Results in Decreased Induction of C5a Secretion by Naturally Occurring Antibodies Against S. aureus

To analyze if the final steps of the complement cascade were also affected by the Fc-binding Fc-III peptide, release of the C5a anaphylatoxin was quantified after opsonization of Wood 46 bacteria with naturally occurring antibodies against S. aureus in NHS in the presence or absence of the peptide.

25 μL NHS (1% final concentration) was pre-incubated with 25 μL Fc-III peptide, control peptide, Fc-III scrambled 2 peptide (20 μg/mL final concentration) or in the absence of peptide (buffer control) in RPMI-HSA for 10 min at RT without shaking and then mixed and incubated with 50 μL Wood 46 bacteria (5×10⁸/mL) in RPMI-HSA for 20 min at 37° C. while shaking at 600 rpm. The bacteria were centrifuged for 7 min at 3500 rpm and supernatant was collected for C5a analysis in a C5a reporter assay described in Bestebroer et al, Cellular Microbiology, 2010 October; 12(10):1506-16. Briefly, human U937 cells stably expressing the C5aR (provided by Eric Prossnitz, University of New Mexico; Kew et al. J Leukoc Biol. 1997 March; 61(3):329-37) were labeled with the cytoplasmic calcium-sensitive fluorescent probe Fluo-3 (Fluo-3, AM; Molecular Probes, Cat #F1241), which exhibits an increase in fluorescence upon binding of Ca²⁺. Activation of the C5aR by C5a binding results in the release of intracellular Ca²⁺. Cells were cultured in RMPI with 10% FCS, washed and resuspended at 1×10⁶ c/mL in RPMI-0.05% HSA. Per sample, 225 μL cells were measured for 9 sec by flow cytometry (FACSVerse; BD) to determine the basal fluorescence intensity of the reporter cells. Subsequently, the reporter cells were stimulated in real time with 25 μL of the supernatants and the change in fluorescence signal was recorded until total time of 50 sec. As a positive control, reporter cells were stimulated with 10⁻⁸M synthetic C5a (C5a Anaphylatoxin (human) trifluoroacetate salt; Bachem, Cat #H-6322) to induce maximal increase in fluorescence signal. C5a generation was calculated relative to the buffer control without peptide, which was set to 100%.

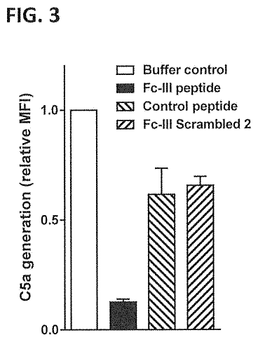

FIG. 3 shows that incubation of Wood 46 bacteria with NHS in the presence of the competing Fc-III peptide resulted in strongly decreased C5a generation relative to the buffer control without peptide (from 100% to 12%), whereas the control peptides only had a minor effect.

In conclusion, inhibition of C5a release by the competing Fc-III peptide DCAWHLGELVWCT suggests that antibodies against S. aureus that are naturally occurring in human sera establish Fc-Fc interactions involved in IgG hexamerization upon binding to non-Protein A-bearing Wood 46 S. aureus to activate final steps in the complement cascade.

### Example 5: Binding of Monoclonal Antibodies Against Two S. aureus Strains

Two recombinant antibodies directed against S. aureus surface molecules were produced as IgG1 as described in Example 1 and tested for binding to several S. aureus strains. IgG1-S4497 recognizes wall teichoic acid (WTA) (Lehar et al., Nature 2015 Nov. 19; 527(7578):323-8) and IgG1-T1-2-F405L (based on tefibazumab) recognizes clumping factor A (CIfA) (Domanski et al., Infect Immun. 2005 August; 73(8):5229-32). Binding was tested by FACS analysis on Wood 46, USA300, 8325-4 (acquired from Prof. T. J. Foster; Trinity College Dublin) and COL (acquired from Prof. Andreas Peschel; University Tubingen). The F405L mutation (EU numbering) related to the generation of bispecific antibodies according to WO2011/131746 (Labrijn et al., Proc Natl Acad Sci USA. 2013 Mar. 26; 110(13):5145-50) and is not relevant for this application. The F405L mutation has been shown before to have no effect on binding of antibodies.

Strains were grown overnight at 37° C. on blood agar, washed with PBS, and resuspended to 5×10⁸ c/mL in HEPES++ with 0.5% BSA. 50 μL bacteria were mixed with 50 μL 2.5 μg/mL antibody (final concentration 1.25 μg/mL) and incubated for 30 min at 4° C. while shaking (600 rpm). Bacteria were washed twice with PBSA and incubated with 50 μL of an Alexa⁶⁴⁷-labelled Protein A (Molecular probes, Cat #P21462) for 45 min at 4° C. while shaking (600 rpm). Samples were washed twice with PBSA, fixed with 150 μL 1% paraformaldehyde in PBS and analyzed by FACS using a FACSVerse apparatus (BD Biosciences).

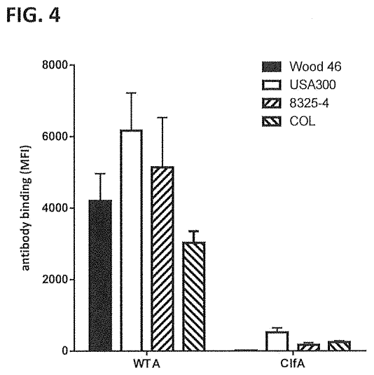

Anti-WTA IgG1-S4497 showed superior binding to all tested S. aureus strains compared to anti-CIfA IgG1-T1-2 (FIG. 4). Therefore, IgG1-S4497 against WTA was selected for further analyses described in Examples 6-8.

### Example 6: Introduction of the Hexamerization Enhancing Mutation E430G in Anti-WTA Antibody IgG1-S4497 Results in Enhanced Complement Deposition and Phagocytic Uptake

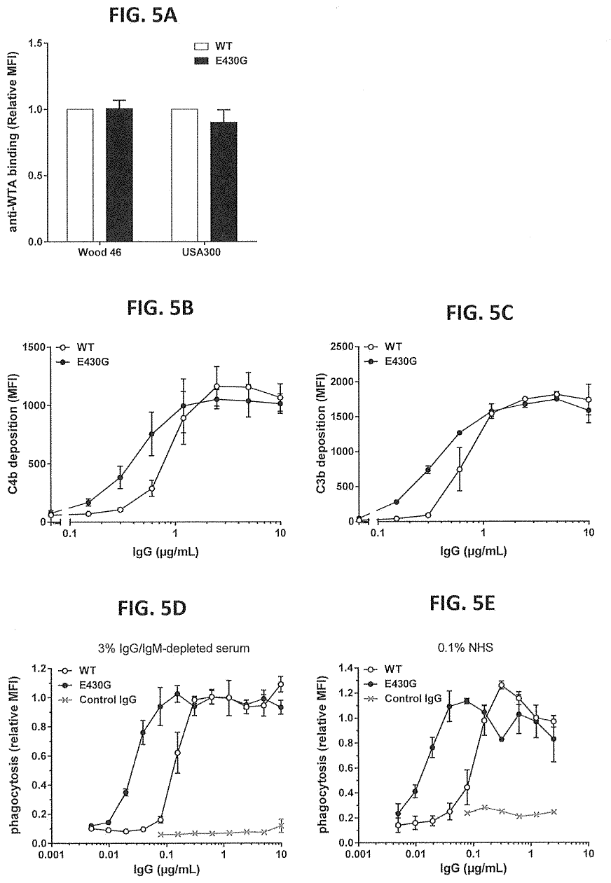

The hexamerization enhancing mutation E430G according to EU numbering was introduced in anti-WTA antibody IgG1-S4497. Binding of IgG1-S4497-E430G to S. aureus strains Wood 46 and USA300 was compared to binding of wild type (WT) IgG1-S4497 in a FACS binding assay as described in Example 5. FIG. 5A shows that binding of IgG1-S4497 to Wood 46 and USA300 was not affected by introduction of the E430G mutation.

The capacity of IgG1-S4497-E430G to induce complement activation on S. aureus was compared to the capacity of WT IgG1-S4497. First, the capacity of the IgG1 antibodies to deposit C4b and C3b was analyzed in a purified classical pathway system. Purified components were used instead of serum to guarantee that no natural antibodies against S. aureus were present that could influence the measurements. Wood 46 bacteria were grown, and collected as described in Example 2. 50 μL of an antibody concentration series (final concentrations 0.08-10 μg/mL in 2-fold dilutions) of IgG1-S4497 or IgG1-S4497-E430G was added to 50 μL washed bacteria at 5×10⁸/mL and incubated for 30 min at 37° C. while shaking (600 rpm). Opsonized bacteria were washed twice with PBSA, resuspended in 50 μL HEPES buffer with 1 μg/mL C1 (Complement Technology, Cat #A098) and incubated for 30 min at 4° C. while shaking at 600 rpm. Samples were washed twice with PBSA, resuspended in 50 μL HEPES buffer with 10 μg/mL C4 (Complement Technology, Cat #A105) and incubated for 30 min at 37° C. while shaking at 600 rpm. For C3b deposition, samples were subsequently also incubated with C2 (final concentration 10 μg/mL, Complement Technology, Cat #A112) together with C3 (final concentration 10 μg/mL; isolated from human plasma according to Rooijakkers and Wu, Nature Immunology, 2009 July; 10(7):721-7) for 30 min at 37° C. while shaking at 600 rpm. Samples were washed and fixed and deposition of C4d and C3d was analyzed in a FACS assay as described in Example 2. Incubation with anti-WTA mAb resulted in dose-dependent C4b and C3b deposition for both IgG1-S4497 and IgG1-S4497-E430G. Remarkably, introduction of the E430G mutation in IgG1-S4497 resulted in enhanced C4b (FIG. 5B) and C3b (FIG. 5C) deposition on Wood 46 bacteria. Data were analyzed in GraphPad Prism (version 6.02) using the nonlinear fit for log(agonist) versus response with variable slope (four parameters). For C4b deposition the EC50 values were 0.886±0.040 μg/mL for IgG1-S4497 and 0.431±0.080 μg/mL for IgG1-S4497-E430G, respectively. For C3b deposition the EC50 values were 0.668±0.031 μg/mL for IgG1-S4497 and 0.354±0.068 μg/mL for IgG1-S4497-E430G, respectively.

Next, the effect of introducing the hexamerization enhancing E430G mutation on the capacity of IgG1-S4497 to induce neutrophil-mediated phagocytosis was analyzed. Wood 46 bacteria were genetically modified with an improved GFP-expressing plasmid pCM29 that constitutively and robustly express the superfolded green fluorescent protein (sGFP) from the sarAP1 promoter (Pang et al., J Innate Immun. 2010; 2(6):546-59). Competent bacteria were electroporated with 10 μL plasmid with a Gene Pulser II (BioRad; 100 Ohm resistance, 25 uF capacitance at 2.5 kV) as described in Schenk et al., FEMS Microbiol Lett. 1992 Jul. 1; 73(1-2):133-8. After recovery, bacteria were selected on Todd Hewitt agar plates containing 10 μg/mL chloramphenicol (Sigma-Aldrich, Cat #C0378). A colony was picked for overnight propagation in 3 mL liquid THB (Oxoid, Cat #CMO189B) with 10 μg/mL chloramphenicol at 37° C. while shaking. Bacteria were washed with PBS, resuspended in 1 mL 15% glycerol in PBS and stored at −80° C. For the phagocytosis experiments, GFP-labeled Wood 46 was grown in 3 mL THB for 18 hours at 37° C. while shaking at 600 rpm, diluted 1:50 into 10 mL fresh THB and subsequently cultured for 3 hours at 37° C. while shaking at 600 rpm. Bacteria were washed twice with PBS, and resuspended at 5×10⁸ c/mL in 1×RPMI 1640 Medium+L-Glutamine+25 mM HEPES (Gibco Life Technologies; Cat #52400) supplemented with 0.05% HSA (Sanquin; Albuman 200 g/L for iv use). 20 μl of 3.75×10⁷/mL GFP-labeled Wood 46 bacteria were opsonized by incubation for 15 min at 37° C. while shaking (600 rpm) with 10 μl of an antibody concentration series of IgG1-S4497, IgG1-S4497-E430G or IgG1-b12 isotype control (0.005-10 μg/mL in 2-fold dilutions) plus 10 μl of either 0.4% NHS (0.1% final serum concentration) or 12% serum devoid of natural IgG and IgM antibodies (3% final serum concentration). Human neutrophils were isolated as described in Example 3. 10 μl of 6.5×10⁶/mL neutrophils were added to the opsonized bacteria and phagocytosis was allowed for 15 min at 37° C. while shaking at 750 rpm. The reaction was stopped with cold paraformaldehyde and fluorescence of the neutrophils was analyzed by flow cytometry as described in Example 3.

For IgG/IgM depletion of NHS, EDTA (500 mM stock in water) was added to pooled NHS to a final concentration of 5 mM and 5 mL serum was run over a 5 mL HiTrap Protein G column (GE Healthcare; Cat #17-0405-01) in tandem with a 5 mL HiTrap NHS-Sepharose column (GE Healthcare; Cat #17-0717-01) coupled with goat-anti-Hu-IgM in the cold with 20 mM sodium phosphate buffer pH 7.5. Collected peak fractions were pooled, reconstituted with 10 mM CaCl₂+10 mM MgCl₂, and aliquots stored at −80° C. The procedure was performed on an AKTA FPLC with fraction collector (GE Healthcare Life Sciences) with ice-cold buffers and columns. Coupling of 2 mg Goat-anti-Hu-IgM (ThermoFisher Scientific; Cat #31136) to the NHS-Sepharose column was performed according to the general protocol provided by GE Healthcare (Instructions 71-7006-00 AX) using alternating 0.5 M ethanolamine with 0.5 M NaCl, pH 8.3 and 0.1 M sodium acetate with 0.5 M NaCl, pH 4 for deactivation of excess reactive groups.

Incubation with anti-WTA mAb resulted in dose-dependent neutrophil-mediated uptake of GFP-labeled bacteria, both in the presence and absence of natural antibodies in the NHS (FIG. 5). Remarkably, introduction of the hexamerization enhancing mutation resulted in enhanced efficacy of neutrophil-mediated pagocytic uptake induced by IgG1-S4497-E430G compared to IgG1-S4497. This increase was observed both in serum devoid of natural antibodies (FIG. 5D) and in normal human serum (FIG. 5E). Data were analyzed as described above resulting in an EC50 of 0.144±0.024 μg/mL for IgG1-S4497 and an EC50 of 0.027±0.060 μg/mL for IgG1-S4497-E430G, respectively using NHS devoid of IgG and IgM. In the presence of intact NHS, the EC50 for IgG1-S4497 was 0.096±0.047 μg/mL and 0.015±0.104 μg/mL for IgG1-S4497-E430G, respectively.

Together, these data indicate that enhancing hexamerization of anti-WTA IgG1-S4497 on the bacterial surface by introduction of the E430G mutation can result in enhanced complement deposition and neutrophil-mediated phagocytic uptake of non-Protein A-bearing S. aureus Wood 46 bacteria.

### Example 7: Complement Deposition on S. aureus after Binding of Anti-WTA IgG1-S4497 Requires Fc-Fc Interactions to Form Antibody Hexamers

To analyze the requirement of antibody hexamer formation by IgG1-S4497 to induce complement deposition on S. aureus, we made use of the self-repulsing mutations K439E and S440K (Diebolder et al., Science. 2014 Mar. 14; 343(6176):1260-3). The Fc repulsion between antibodies that is introduced by the presence of either K439E or S440K in an IgG1 antibody results in inhibition of hexamerization (WO2013/0044842). The repulsion by the K439E and S440K mutations is neutralized by combining both mutations in a mixture of two antibodies each harboring one and the other mutation, resulting in restoration of the Fc-Fc interactions and hexamerization.

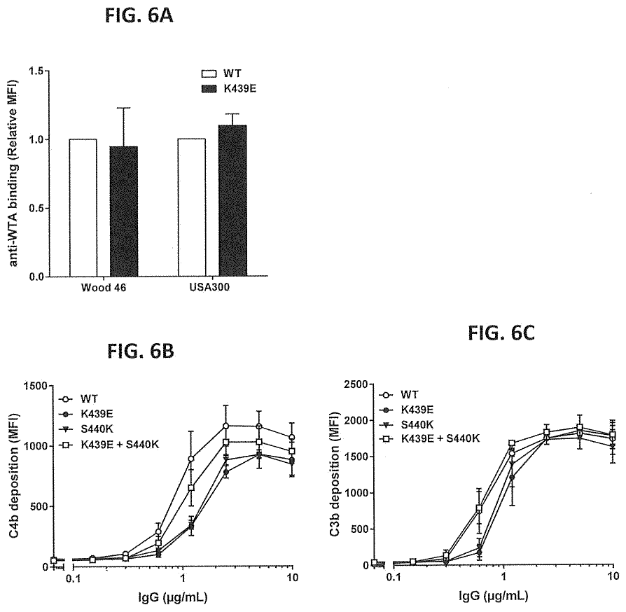

First, binding of IgG1-S4497-K439E to S. aureus strains Wood 46 and USA300 was compared to binding of WT IgG1-S4497 in a FACS binding assay as described in Example 5. FIG. 6A shows that binding of IgG1-S4497 to Wood 46 and USA300 was not affected by introduction of the K439E mutation.

Next, the capacity of IgG1-S4497-K439E, IgG1-S4497-S440K and the combination of the two antibodies to deposit C4b and C3b on Wood 46 was tested in a purified classical pathway system as described in Example 6. For the inhibiting variants K439E and S440K, reduced C4b (FIG. 6B) and C3b (FIG. 6C) deposition was observed compared to WT IgG1-S4497. However, when repulsion was neutralized by combining the two antibodies each having one of the complementary mutations K439E and S440K, C4b (FIG. 6B) and C3b (FIG. 6C) deposition was restored to WT levels. EC50 values were calculated as described in Example 6. For C4b deposition the EC50 values were 0.886±0.040 μg/mL for WT IgG1-S4497, 1.490±0.028 μg/mL for IgG1-S4497-K439E, 1.354±0.033 μg/mL for IgG1-S4497-S440K, and 1.034±0.033 μg/mL for the combination IgG1-S4497-K439E+IgG1-S4497-S440K. For C3b deposition the EC50 values were 0.668±0.031 μg/mL for WT IgG1-S4497, 1.035±0.031 μg/mL for IgG1-S4497-K439E, 0.899±0.036 μg/mL for IgG1-S4497-S440K, and 0.657±0.025 μg/mL for the combination IgG1-S4497-K439E+IgG1-S4497-S440K.

These data indicate that antibody hexamerization by Fc-Fc interactions can improve C4b and C3b deposition on S. aureus Wood 46 by anti-WTA IgG1-S4497.

### Example 8: IgG2-S4497 Against S. aureus Antigen WTA Induces Complement-Dependent Phagocytic Uptake by Human Neutrophils, which can be Enhanced by Introduction of the Hexamerization Enhancing Mutation E430G

Since naturally occurring antibodies against S. aureus antigen WTA predominantly include IgG2 (Jung et al., J Immunol. 2012 Nov. 15; 189(10):4951-9), we also tested the capacity of the anti-WTA antibody in an IgG2 backbone to induce phagocytic uptake of GFP-labeled Wood 46 bacteria by freshly isolated human neutrophils as described in Example 6. IgG2-S4497 and IgG2-S4497-E430G were generated as described in Example 1. The effect of introducing the hexamerization enhancing mutation E430G in IgG2-S4497 on phagocytic uptake was tested in the presence of serum. Furthermore, the capacity of WT IgG2-S4497 to induce phagocytic uptake was compared to IgG1-S4497, both in the presence and absence of serum, and thus complement. Data were analyzed in GraphPad Prism and EC50 values were calculated as described in Example 6.

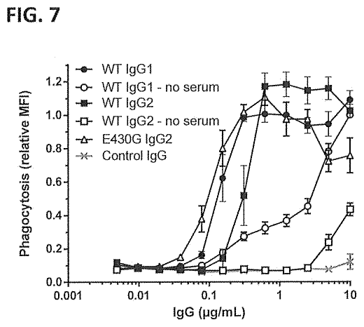

FIG. 7 shows that in the absence of serum, WT IgG1-S4497 antibody was able to induce more potent phagocytic uptake than WT IgG2-S4497 antibody (EC50 3.303±0.04 μg/mL and 16±0.038 μg/mL, respectively). Remarkably, addition of serum and thus complement had a strong effect on both WT IgG1-S4497 and WT IgG2-S4497 (EC50 0.130±0.039 μg/mL and 0.331±0.061 μg/mL, respectively), which does not support the assumption that the IgG2 subclass is inefficient in complement activation.

IgG2-S4497-E430G induced enhanced phagocytic uptake compared to WT IgG2-S4497 (EC50 0.107±0.073 μg/mL and 0.331±0.061 μg/mL, respectively) indicating that introduction of the hexamerization enhancing mutation in the anti-WTA IgG2 antibody potentiated the induction of neutrophil-mediated phagocytosis in the presence of complement. In the presence of serum, the levels of phagocytic uptake for IgG2-S4497-E430G were comparable to WT IgG1-S4497 (EC50 0.107±0.073 μg/mL and 0.130±0.039 μg/mL, respectively).

### Example 9: Introduction of the Hexamerization Enhancing Mutation E430G in an Anti-CP5 Monoclonal IgG1 Antibody Results in Enhanced Complement Deposition and Phagocytic Uptake

S. aureus surface molecules such as WTA can be shielded from recognition by antibodies by expression of a polysaccharide (PS) capsule. Therefore, a monoclonal antibody against capsular polysaccharide type 5 (CP5) was generated as WT IgG1-CP5 and IgG1-CP5-E430G as described in Example 1 and tested for binding and complement activation on S. aureus bacteria.

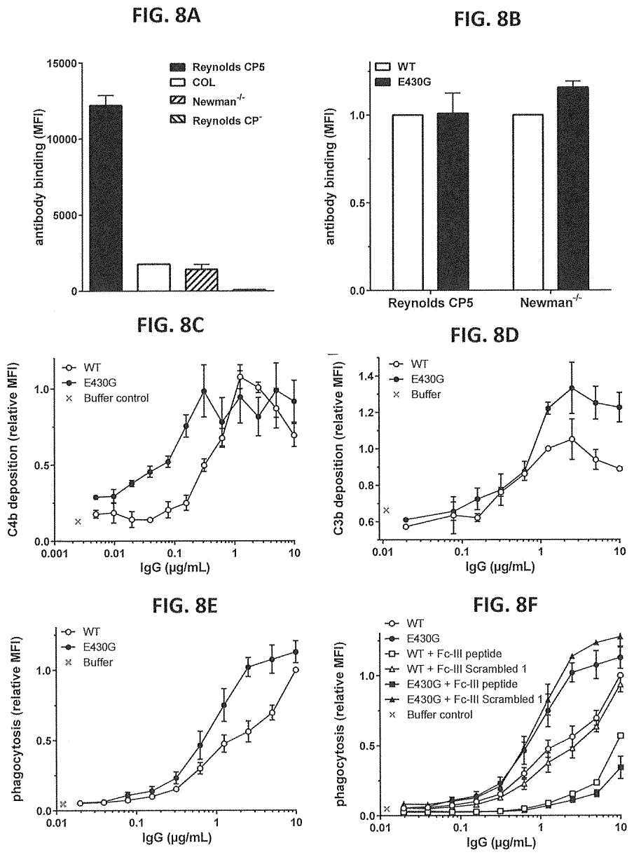

Binding of WT IgG1-CP5 to several CP5-encapsulated S. aureus strains was tested in a FACS binding assay as described in Example 5. Binding was tested on Reynolds CP5 (provided by Dr. Jeane Lee, Brigham and Women's Hospital, Boston), Reynolds CP⁻ (provided by Dr. Jeane Lee, Brigham and Women's Hospital, Boston), COL (provided by Prof. Andreas Peschel, University Tubingen) and Newman^{-/-} (provided by Prof. Tim Foster, Trinity College, Dublin). IgG1-CP5 showed strong binding to the Reynolds CP5 strain that is known to express high levels of CP5 (FIG. 8A). Reynolds CP5 was used for further analyses. First it was shown that introduction of the E430G mutation in IgG1-CP5 did not affect binding of IgG1-CP5 to Reynolds CP5 (FIG. 8B).

The capacity of IgG1-CP5-E430G to induce complement activation on S. aureus was compared to the capacity of WT IgG1-CP5. Since serum of healthy individuals does not contain high concentrations of naturally occurring antibodies against capsular polysaccharides, all experiments were performed in the presence of NHS.

First, the effect of the E430G mutation on the capacity of IgG1-CP5 to deposit C4b and C3b on bacteria was analyzed. Reynolds CP5 bacteria were grown and collected as described in Example 2. For opsonization, 50 μL washed bacteria (5×10⁷/mL) were added to 50 μL of an antibody concentration series (final concentrations 0.08-10 μg/mL in 2-fold dilutions) of IgG1-CP5 or IgG1-CP5-E430G in 1% pooled NHS and incubated for 20 min at 37° C. while shaking at 600 rpm. Bacteria were washed twice with PBSA and deposited C4d and C3b was stained and analyzed by FACS as described in Example 2. Incubation with anti-CP5 mAb resulted in dose-dependent C4b and C3b deposition for both IgG1-CP5 and IgG1-CP5-E430G. Both C4b (FIG. 8C) and C3b (FIG. 8D) deposition were enhanced with the E430G variant IgG1-CP5-E430G compared to the WT IgG1-CP5 antibody.

Next, the effect of the hexamerization enhancing E430G mutation on the capacity of IgG1-CP5 to induce neutrophil-mediated phagocytic uptake was analyzed. Reynolds CP5 bacteria were GFP-labeled and incubated with WT IgG1-CP5 and IgG1-CP5-E430G in pooled NHS serum as described in Example 6. 20 μL of 3.75×10⁷/mL GFP-labeled Reynolds CP5 bacteria were opsonized by incubation for 15 min at 37° C. while shaking at 600 rpm with 20 μL of an antibody concentration series of IgG1-CP5 or IgG1-CP5-E430G (final concentrations 0.005-10 μg/mL in 2-fold dilutions) in NHS (3% final concentration). Next, 10 μL of 7.5×10⁶/mL neutrophils were added to the opsonized bacteria and phagocytic uptake was allowed for 15 min at 37° C. while shaking at 750 rpm. The reaction was stopped with cold paraformaldehyde and fluorescence of the neutrophils was analyzed by flow cytometry as described in Example 3.

Incubation with anti-CP5 mAb resulted in dose-dependent neutrophil-mediated uptake of GFP-labeled bacteria, indicating that addition of anti-CP5 antibodies could neutralize the anti-phagocytic activity of the capsule (FIG. 8E). Remarkably, an increased phagocytic uptake was observed with IgG1-CP5-E430G compared to WT IgG1-CP5. In another phagocytic uptake experiment, the antibody concentration series in serum was pre-incubated for 10 min at RT with Fc-III peptide or Fc-III Scrambled 1 peptide (10 μL of antibody concentration series at 0.02-40 μg/mL in 2-fold dilutions+10 μL of 12% serum with 40 μg/mL peptide). The Fc-III peptide was able to potently inhibit phagocytic uptake by both the WT and E430G variant of the anti-CP5 antibody (FIG. 8F).

Together, these data indicate that the tested anti-CP5 antibody could induce hexamerization-dependent complement-mediated phagocytic uptake of the encapsulated S. aureus strain Reynolds CP5, which could be increased by the hexamerization enhancing mutation E430G.

### Example 10: Introduction of the Hexamerization Enhancing Mutation E430G in Anti-WTA Antibody IgG1-S4497 Results in Enhanced Phagocytic Kill of S. aureus

The capacity of IgG1-S4497-E430G to induce phagocytic kill of Wood 46 S. aureus bacteria was compared to the capacity of WT IgG1-S4497. Wood 46 bacteria were grown in 3 mL Todd Hewitt Broth (THB, Oxoid, Cat #CMO189) with Yeast Extract (Oxoid, Cat #LP0021) for 18 hrs at 37° C. while shaking at 600 rpm, diluted 1/100 in fresh THB and grown to OD660 ^{˜}0.50. Bacteria were washed twice with Hank's Balanced Salt Solution (HBSS, without phenol red; Lonza, Cat #BE10-527F), and adjusted to a concentration of 1.7×10⁸ bacteria/mL in HBSS+0.1% HSA. 20 μL of 1.7×10⁸/mL Wood 46 bacteria were opsonized by incubation for 5 min at 37° C. while shaking (700 rpm) with 10 μL of an antibody concentration series of IgG1-S4497 or IgG1-S4497-E430G (starting at 3 or 1 μg/mL in 3- or 2-fold dilutions) plus 10 μL IgG-depleted serum (1% final serum concentration). IgG depletion of NHS was performed as described in Example 6. Human neutrophils were freshly isolated under sterile conditions as described in Example 3 and adjusted to a concentration of 1×10⁷ cells/mL in HBSS+0.1% HSA. 85 μL of 1×10⁷/mL neutrophils were added to 15 μL of the opsonized bacteria in sterile siliconized 2 mL tubes (Sigma-Aldrich, Cat #T3531), and phagocytosis was allowed for 90 min at 37° C. in a CO2-incubator on a shaking platform (750 rpm). Final ratio of bacteria to neutrophils was 1:1 with 1% serum. The reaction was stopped with 900 μL 0.3% Saponin (Sigma-Aldrich, Cat #47036) in water, vortexed and incubated for 10 minutes on ice. Appropriate dilutions in PBS were prepared and 25 μL drops placed on Todd Hewitt Agar plates in duplicate and incubated overnight at 37° C. Colony forming units (CFU) were counted and expressed relative to the bacteria only sample without antibody or neutrophils.

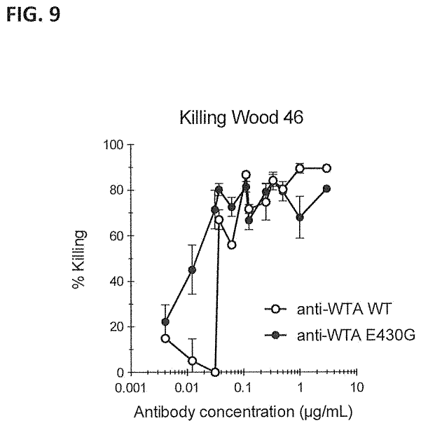

Incubation with anti-WTA mAb resulted in dose-dependent neutrophil-mediated killing of bacteria (FIG. 9). Remarkably, introduction of the hexamerization enhancing mutation resulted in enhanced efficacy of neutrophil-mediated killing induced by IgG1-S4497-E430G compared to IgG1-S4497. The EC50 was 0.037±0.074 μg/mL for IgG1-S4497 and 0.013±0.071 μg/mL for IgG1-S4497-E430G, respectively.

### Example 11: Introduction of the Hexamerization Enhancing Mutation E430G in Anti-WTA Antibody IgG1-S4497 Results in Enhanced Phagocytic Uptake of Staphylococcus warneri

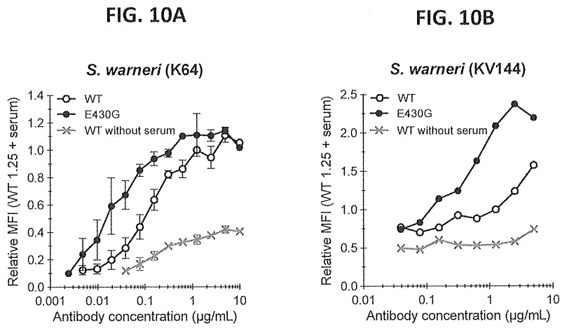

Bacteria of the S. warneri strains K64 and KV144 (both clinical isolates from the Department of Medical Microbiology of the University Medical Center Utrecht (UMCU)) were FITC-labeled as described in Example 3. 20 μL of 3.75×10′/mL FITC-labeled S. warneri K64 and KV144 bacteria were opsonized by incubation for 15 min at 37° C. while shaking (600 rpm) with 10 μL of an antibody concentration series of IgG1-S4497 or IgG1-S4497-E430G (0.002-5 μg/mL in 2-fold dilutions) plus 10 μL 4% NHS (1% final serum concentration). Human neutrophils were isolated as described in Example 3. 10 μL of 6.5×10⁶/mL neutrophils were added to the opsonized bacteria and phagocytosis was allowed for 15 min at 37° C. while shaking at 750 rpm. The reaction was stopped with cold paraformaldehyde and fluorescence of the neutrophils was analyzed by flow cytometry as described in Example 3. Incubation with anti-WTA antibody IgG1-S4497 resulted in dose-dependent neutrophil-mediated uptake of FITC-labeled S. warneri bacteria (FIG. 10). Remarkably, introduction of the hexamerization enhancing mutation E430G resulted in enhanced efficacy of neutrophil-mediated phagocytic uptake induced by IgG1-S4497-E430G compared to IgG1-S4497. The EC50 values on S. warneri strain K64 was 0.126±0.096 μg/mL for IgG1-S4497 and 0.016±0.395 μg/mL for IgG1-S4497-E430G, respectively.

### Example 12: Introduction of the Hexamerization Enhancing Mutation E430G in Anti-WTA Antibodies IgG1-6297 Results in Enhanced Complement Deposition

The hexamerization enhancing mutation E430G according to EU numbering was introduced in anti-WTA antibody IgG1-6297. The capacity of IgG1-6297-E430G to induce complement activation on S. aureus was compared to the capacity of WT IgG1-6297. The capacity of the IgG1 antibodies to deposit C1q and C4b on bacteria was analyzed. S. aureus COL bacteria were genetically modified to express GFP and grown and collected as described in Example 6. Bacteria were grown overnight on Sheep Blood Agar plates and collected into PBS as described in Example 5. For opsonization, 20 μL washed bacteria (5×10⁷/mL) in RPMI/HSA buffer were added to 20 μL of an antibody concentration series of IgG1-6297 or IgG1-6297-E430G in 1% pooled IgG/IgM-depleted serum and incubated for 30 min at 37° C. while shaking at 750 rpm. Bacteria were washed twice with RPMI/HSA and deposited C4b was detected with 1 μg/mL of mouse-anti-human C4d antibody (Quidel, Cat #A213) and an Allophycocyanin (APC)-conjugated goat anti-mouse Immunoglobulins antibody (1:350 dilution; BD Pharmingen, #550826), essentially as described in Example 2. For C1q detection, antibody incubations were performed with a FITC-conjugated rabbit anti-C1q antibody (1:350 dilution, Dako, Cat #F0254) and an APC-conjugated goat F(ab′)₂ anti-rabbit-IgG(H+L) (1:350 dilution, Jackson Immunoresearch, Cat #111-136-144). Fluorescence was measured by flow cytometry.

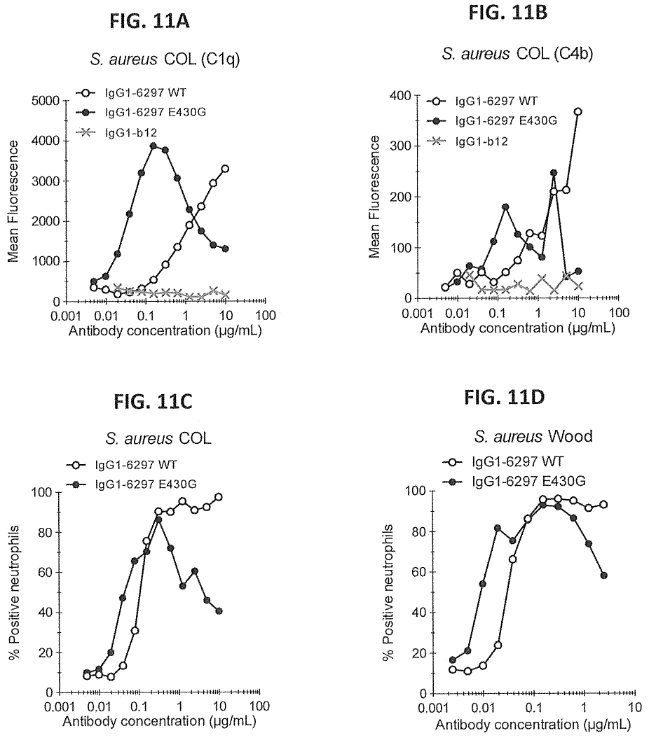

To analyze the phagocytic uptake of S. aureus bacteria by IgG1-6297 and IgG1-6297-E430G, 20 μL of 3.75×10⁷/mL GFP-expressing COL or FITC-labeled Wood 46 bacteria were opsonized by incubation for 15 min at 37° C. while shaking (750 rpm) with 10 μL of an antibody concentration series of IgG1-56297 or IgG1-56297-E430G (0.002-5 μg/mL in 2-fold dilutions) plus 10 μL 4% IgG-depleted NHS (1% final serum concentration). Human neutrophils were isolated as described in Example 3. 10 μL of 6.5×10⁶/mL neutrophils were added to the opsonized bacteria and phagocytosis was allowed for 15 min at 37° C. while shaking at 750 rpm. The reaction was stopped with cold paraformaldehyde and fluorescence of the neutrophils was analyzed by flow cytometry as described in Example 3. FIG. 11 shows that Incubation with 6297 mAb resulted in dose-dependent C1q and C4b deposition for both IgG1-6297 and IgG1-6297-E430G. Both C1q (FIG. 11A) and C4b (FIG. 11B) deposition were enhanced with the E430G variant IgG1-6297-E430G compared to the WT IgG1-6297 antibody. Also the neutrophil-mediated phagocytic uptake by IgG1-6297-E430G was enhanced compared to WT IgG1-6297 on GFP-labeled COL (FIG. 11C) and FITC-labeled Wood 46 (FIG. 11D) cells.

1. An antibody comprising an Fc region of a human immunoglobulin IgG and an antigen binding region binding to wall teichoic acid (WTA) or capsular polysaccharides (CP), wherein the Fc region comprises a mutation of an amino acid at a position corresponding to E430, E345 or S440 in human IgG1 according to EU numbering.

2. The antibody according to claim 1, wherein the mutation is selected from the group consisting of E430G, E345K, E430S, E430F, E430T, E345Q, E345R, E345Y, S440W and S440Y.

3. (canceled)

4. The antibody according to claim 1, wherein the antibody further comprises a mutation selected form the group of K439E and S440K.

5. The antibody according to claim 1, wherein the WTA is WTA-alpha or WTA-beta.

6. The antibody according to claim 1, wherein the CP is CP5.

7. The antibody according to claim 1, wherein the antibody is an IgG1, IgG2, IgG3, IgG4, IgE, IgD or IgM isotype.

8. (canceled)

9. The antibody according to claim 1, wherein the antibody is a monoclonal antibody.

10. The antibody according to claim 1, wherein the antibody is a mammal, human, or ungulate antibody.

11. The antibody according to claim 1, wherein the antibody is a humanized or chimeric antibody.

12. The antibody according to claim 1, wherein the antibody enhances phagocytosis or complement activation.

13-16. (canceled)

17. A composition comprising the antibody according to claim 1 and a carrier.

18-19. (canceled)

20. A composition comprising first and second antibodies according to claim 1.

21. The composition according to claim 20, wherein the first antibody further comprises an K439E mutation and the second antibody further comprises a S440K mutation or wherein the first antibody further comprises a S440K mutation and the second antibody further comprises an E439E mutation.

22. (canceled)

23. The composition according to claim 20, wherein the composition comprises a first antibody which binds WTA alpha and the second antibody which binds WTA beta or vice versa.

24. The composition according to claim 20, wherein the composition comprises a first antibody which binds WTA and the second antibody which binds CP or vice versa.

25-27. (canceled)

28. A method of treating an infection caused by gram positive bacteria comprising administering to a subject in need thereof an effective amount of the antibody of claim 1, or a composition comprising the antibody.

29. (canceled)

30. A method of treating an infection caused by Staphylococcus aureus, MRSA or MSSA comprising administering to a subject in need thereof an effective amount of the antibody of claim 1, or a composition comprising the antibody.

31. A method of treating an infection caused by Staphylococcus warneri comprising administering to a subject in need thereof an effective amount of the antibody of claim 1, or a composition comprising the antibody.

32. A method of treating surgical site infections, wound infections, cystic fibrosis, pneumonia, ventilator-associated pneumonia, sepsis, toxic shock syndrome, intravenous line infections, and infections in the presence of prosthetic devices comprising administering to a subject in need thereof an effective amount of the antibody of claim 1, or a composition comprising the antibody.

33. A method of enhancing the effector function of an antibody comprising an Fc region and an antigen binding region binding to wall teichoic acid (WTA) or capsular polysaccharides (CP), the method comprising introducing a mutation in the Fc region corresponding to position E430, E345 or S440 in human IgG1, wherein the numbering of positions is according to EU numbering.

34. The method according to claim 33, wherein the Fc region comprises a mutation selected from E430G, E345K, E430S, E430F, E430T, E345Q, E345R, E345Y, S440W or S440Y.

35-40. (canceled)

41. A method of enhancing Fc-Fc contact between antibody molecules on a target cell in vivo comprising:
i) providing an antibody according to claim 1,
ii) bringing the antibody in contact with the antigen on the cell surface of Gram-positive bacteria under in vivo conditions, and
iii) in a concentration that allows Fc-Fc interaction.

42-44. (canceled)
